(19) United States
(12) Patent Application Publication
(10) Pub. No.: US 2021/0277071 A1
(43) Pub. Date: Sep. 9, 2021

(54) HYBRID NEUROTOXINS

(71) Applicant: IPSEN BIOPHARM LIMITED, Wrexham (GB)

(72) Inventors: Elena FONFRIA-SUBIROS, Abingdon (GB); David BURGIN, Abingdon (GB)

(21) Appl. No.: 16/325,046

(22) PCT Filed: Sep. 28, 2017
(86) PCT No.: PCT/EP2017/074665

§ 371 Date: Feb. 12, 2019

(30) Foreign Application Priority Data
Sep. 29, 2016 (EP) 16191468.4

(51) Int. Cl. C07K 14/33; C07K 14/28; C07K 14/245

(52) U.S. Cl. CPC C07K 14/33; C07K 14/28; A61K 38/00; C07K 2319/55; C07K 14/245

(57) ABSTRACT

The present invention relates to hybrid neurotoxins comprising a clostridial light chain and a selective ganglioside binding moiety and to their use in therapy.

### FIELD OF THE INVENTION

The present invention relates to hybrid neurotoxins with improved therapeutic properties, in particular a more selective binding affinity for gangliosides.

### BACKGROUND

Bacteria in the genus Clostridia produce highly potent and specific protein toxins, which can poison neurons and other cells to which they are delivered. Examples of such clostridial toxins include the neurotoxins produced by C. tetani (TeNT) and by C. botulinum (BoNT) serotypes A-G, as well as those produced by C. baratii and C. butyricum.

Among the clostridial neurotoxins are some of the most potent toxins known. By way of example, botulinum neurotoxins have median lethal dose (LD₅₀) values for mice ranging from 0.5 to 5 ng/kg, depending on the serotype. Both tetanus and botulinum toxins act by inhibiting the function of affected neurons, specifically the release of neurotransmitters. While botulinum toxin acts at the neuromuscular junction and inhibits cholinergic transmission in the peripheral nervous system, tetanus toxin acts in the central nervous system.

In nature, clostridial neurotoxins are synthesised as a single-chain polypeptide that is modified post-translationally by a proteolytic cleavage event to form two polypeptide chains joined together by a disulphide bond. Cleavage occurs at a specific cleavage site, often referred to as the activation site, that is located between the cysteine residues that provide the inter-chain disulphide bond. It is this dichain form that is the active form of the toxin. The two chains are termed the heavy chain (H-chain), which has a molecular mass of approximately 100 kDa, and the light chain (L-chain), which has a molecular mass of approximately 50 kDa. The H-chain comprises an N-terminal translocation component (H_{N} domain) and a C-terminal targeting component (H_{C} domain). The cleavage site is located between the L-chain and the translocation domain components.

Following binding of the H_{C} domain to its target neuron and internalisation of the bound toxin into the cell via an endosome, the H_{N} domain translocates the L-chain across the endosomal membrane and into the cytosol, and the L-chain provides a protease function (also known as a non-cytotoxic protease).

Non-cytotoxic proteases act by proteolytically cleaving intracellular transport proteins known as SNARE proteins (e.g. SNAP-25, VAMP, or syntaxin)—see Gerald K (2002) “Cell and Molecular Biology” (4th edition) John Wiley & Sons, Inc. The acronym SNARE derives from the term Soluble NSF Attachment Receptor, where NSF means N-ethylmaleimide-Sensitive Factor. SNARE proteins are integral to intracellular vesicle fusion, and thus to secretion of molecules via vesicle transport from a cell. The protease function is a zinc-dependent endopeptidase activity and exhibits a high substrate specificity for SNARE proteins. Accordingly, once delivered to a desired target cell, the non-cytotoxic protease is capable of inhibiting cellular secretion from the target cell. The L-chain proteases of clostridial neurotoxins are non-cytotoxic proteases that cleave SNARE proteins. The L-chain proteases of BoNT/B, BoNT/D, BoNT/F and BoNT/G cleave VAMP, the L-chain proteases of BoNT/A and BoNT/E cleave SNAP25 and the L-chain protease of BoNT/C cleaves both SNAP25 and syntaxin, which result in the inhibition of neurotransmitter release and consequent neuroparalysis (Rossetto, O. et al., “Botulinum neurotoxins: genetic, structural and mechanistic insights.” Nature Reviews Microbiology 12.8 (2014): 535-549).

In view of the ubiquitous nature of SNARE proteins, clostridial neurotoxins such as botulinum toxin have been successfully employed in a wide range of therapies. Currently all approved drugs/cosmetic preparations comprising BoNTs contain naturally occurring neurotoxins purified from clostridial strains (BoNT/A in the case of DYSPORT®, BOTOX® or XEOMIN®, and BoNT/B in the case of MYOBLOC®).

By way of example, we refer to William J. Lipham, Cosmetic and Clinical Applications of Botulinum Toxin (Slack, Inc., 2004), which describes the use of clostridial neurotoxins, such as botulinum neurotoxins (BoNTs), BoNT/A, BoNT/B, BoNT/C1, BoNT/D, BoNT/E, BoNT/F and BoNT/G, and tetanus neurotoxin (TeNT), to inhibit neuronal transmission in a number of therapeutic and cosmetic or aesthetic applications—for example, marketed botulinum toxin products are currently approved as therapeutics for indications including focal spasticity, upper limb spasticity, lower limb spasticity, cervical dystonia, blepharospasm, hemifacial spasm, hyperhidrosis of the axillae, chronic migraine, neurogenic detrusor overactivity, glabellar lines, and severe lateral canthal lines. In addition, clostridial neurotoxin therapies are described for treating neuromuscular disorders (see U.S. Pat. No. 6,872,397); for treating uterine disorders (see US 2004/0175399); for treating ulcers and gastroesophageal reflux disease (see US 2004/0086531); for treating dystonia (see U.S. Pat. No. 6,319,505); for treating eye disorders (see US 2004/0234532); for treating blepharospasm (see US 2004/0151740); for treating strabismus (see US 2004/0126396); for treating pain (see U.S. Pat. Nos. 6,869,610, 6,641,820, 6,464,986, and 6,113,915); for treating fibromyalgia (see U.S. Pat. No. 6,623,742, US 2004/0062776); for treating lower back pain (see US 2004/0037852); for treating muscle injuries (see U.S. Pat. No. 6,423,319); for treating sinus headache (see U.S. Pat. No. 6,838,434); for treating tension headache (see U.S. Pat. No. 6,776,992); for treating headache (see U.S. Pat. No. 6,458,365); for reduction of migraine headache pain (see U.S. Pat. No. 5,714,469); for treating cardiovascular diseases (see U.S. Pat. No. 6,767,544); for treating neurological disorders such as Parkinson's disease (see U.S. Pat. Nos. 6,620,415, 6,306,403); for treating neuropsychiatric disorders (see US 2004/0180061, US 2003/0211121); for treating endocrine disorders (see U.S. Pat. No. 6,827,931); for treating thyroid disorders (see U.S. Pat. No. 6,740,321); for treating cholinergic influenced sweat gland disorders (see U.S. Pat. No. 6,683,049); for treating diabetes (see U.S. Pat. Nos. 6,337,075, 6,416,765); for treating a pancreatic disorder (see U.S. Pat. Nos. 6,261,572, 6,143,306); for treating cancers such as bone tumors (see U.S. Pat. Nos. 6,565,870, 6,368,605, 6,139,845, US 2005/0031648); for treating optic disorders (see U.S. Pat. Nos. 6,358,926, 6,265,379); for treating autonomic disorders such as gastrointestinal muscle disorders and other smooth muscle dysfunction (see U.S. Pat. No. 5,437,291); for treatment of skin lesions associated with cutaneous cell-proliferative disorders (see U.S. Pat. No. 5,670,484); for management of neurogenic inflammatory disorders (see U.S. Pat. No. 6,063,768); for reducing hair loss and stimulating hair growth (see U.S. Pat. No. 6,299,893); for treating downturned mouth (see U.S. Pat. No. 6,358,917); for reducing appetite (see US 2004/40253274); for dental therapies and procedures (see US 2004/0115139); for treating neuromuscular disorders and conditions (see US 2002/0010138); for treating various disorders and conditions and associated pain (see US 2004/0013692); for treating conditions resulting from mucus hypersecretion such as asthma and COPD (see WO 00/10598); and for treating non-neuronal conditions such as inflammation, endocrine conditions, exocrine conditions, immunological conditions, cardiovascular conditions, bone conditions (see WO 01/21213). All of the above publications are hereby incorporated by reference in their entirety.

However, native BoNTs do not discriminate amongst the spatial distribution of neuromuscular junctions or different types of neurons, which can result in side effects. For instance, treatment of upper limb spasticity with BoNT may result in adverse events such as dry mouth and dysphagia (Nair, K. P., and Jonathan Marsden. “The management of spasticity in adults.” Bmj 349 (2014): g4737.)

Increasing the specificity of clostridial neurotoxins for particular neuronal populations would allow to tailor the clostridial neurotoxin to specific pathologies with increased safety and reduced side effects.

The present invention provides neurotoxins with improved therapeutic properties, in particular a more selective binding affinity for specific neurons driving muscle contractions (neuromuscular junction) or cholinergic secretions.

### SUMMARY OF INVENTION

In a first aspect, the present invention provides a hybrid neurotoxin comprising a clostridial light chain (L) and a selective ganglioside binding moiety (GBM), wherein the selective ganglioside binding moiety is not a clostridial H_{CC} or H_{C} domain.

In another aspect, the present invention provides a nucleotide sequence encoding a hybrid neurotoxin according to the invention.

In another aspect, the present invention provides a vector comprising a nucleotide sequence according to the invention.

In another aspect, the present invention provides a cell comprising a nucleotide sequence or a vector according to the invention.

In another aspect, the present invention provides a pharmaceutical composition comprising a hybrid neurotoxin according to the invention.

In another aspect, the present invention provides a hybrid neurotoxin or a pharmaceutical composition according to the invention for use in therapy.

In another aspect, the present invention provides the non therapeutic use of a hybrid neurotoxin or a pharmaceutical composition according to the invention for treating an aesthetic or cosmetic condition.

In another aspect, the present invention provides a hybrid neurotoxin comprising a clostridial light chain and a selective ganglioside binding moiety, wherein the selective ganglioside binding moiety is not a clostridial H_{CC} or H_{C} domain, and wherein said selective ganglioside binding moiety binds to one or more gangliosides selected from GM1a, GM1b, GD1a and GalNAc-GD1a, for use in treating a limb disorder associated with unwanted neuronal activity.

In another aspect, the present invention provides a hybrid neurotoxin comprising a clostridial light chain and a selective ganglioside binding moiety, wherein the selective ganglioside binding moiety is not a clostridial H_{CC} or H_{C} domain, and wherein said selective ganglioside binding moiety binds to one or more gangliosides selected from GT1a and GQ1b, for use in treating a head or neck disorder associated with unwanted neuronal activity.

In another aspect, the present invention provides a method of treatment comprising the administration of a therapeutically effective amount of a hybrid neurotoxin or a pharmaceutical composition according to the invention to a patient in need thereof.

In another aspect, the invention provides a hybrid neurotoxin comprising a clostridial light chain and a selective ganglioside binding moiety, wherein the selective ganglioside binding moiety is not a clostridial H_{CC} or H_{C} domain, and wherein said selective ganglioside binding moiety binds to GM1 for use in treating sialorrhea (or excessive salivation or drooling).

In another aspect, the invention provides a hybrid neurotoxin comprising a clostridial light chain and a selective ganglioside binding moiety, wherein the selective ganglioside binding moiety is not a clostridial H_{CC} or H_{C} domain, and wherein said selective ganglioside binding moiety binds to one or more gangliosides selected from NeuAc GM3, NeuGc GM3, GM2, GM1, GD3 and GD2, for use in treating cancer.

In another aspect, the present invention provides a method of treatment of a limb disorder associated with unwanted neuronal activity, comprising the administration of a therapeutically effective amount of a hybrid neurotoxin comprising a clostridial light chain and a selective ganglioside binding moiety, wherein the selective ganglioside binding moiety is not a clostridial H_{CC} or H_{C} domain, and wherein said selective ganglioside binding moiety binds to one or more gangliosides selected from GM1a, GM1b, GD1a and GalNAc-GD1a to a patient in need thereof.

In another aspect, the present invention provides a method of treatment of a head or neck disorder associated with unwanted neuronal activity, comprising the administration of a therapeutically effective amount of a hybrid neurotoxin comprising a clostridial light chain and a selective ganglioside binding moiety, wherein the selective ganglioside binding moiety is not a clostridial H_{CC} or H_{C} domain, and wherein said selective ganglioside binding moiety binds to one or more gangliosides selected from GT1a and GQ1b to a patient in need thereof.

In another aspect, the present invention provides a method of treating sialorrhea (or excessive salivation or drooling), comprising the administration of a therapeutically effective amount of a hybrid neurotoxin comprising a clostridial light chain and a selective ganglioside binding moiety, wherein the selective ganglioside binding moiety is not a clostridial H_{CC} or H_{C} domain, and wherein said selective ganglioside binding moiety binds to GM1 to a patient in need thereof.

In another aspect, the present invention provides a method of treating cancer, comprising the administration of a therapeutically effective amount of a hybrid neurotoxin comprising a clostridial light chain and a selective ganglioside binding moiety, wherein the selective ganglioside binding moiety is not a clostridial H_{CC} or H_{C} domain, and wherein said selective ganglioside binding moiety binds to one or more gangliosides selected from NeuAc GM3, NeuGc GM3, GM2, GM1, GD3 and GD2 to a patient in need thereof.

### DETAILED DESCRIPTION OF THE INVENTION

The present invention is based on the finding by the inventors that it is possible to alter the selectivity of a clostridial neurotoxin for neuromuscular junctions by engineering exogenous (non clostridial) ganglioside binding domains into the clostridial neurotoxin.

Botulinum neurotoxins (BoNTs) target neurons using a dual receptor binding mechanism involving protein receptors and plasma membrane gangliosides. BoNT/B, /G, and/DC have been shown to recognize the luminal domain of SytI and SytII (the two major isoforms of synaptotagmin). Synaptic vesicle glycoprotein 2 (SV2), including three isoforms SV2A, SV2B, and SV2C, have been shown to be the protein receptors for BoNT/A, BoNT/D, BoNT/E, BoNT/F.

Ganglio sides are oligoglycosylceramides derived from lactosylceramide and containing a sialic acid residue such as N-acetylneuraminic acid (NANA′ or ‘SA’ or ‘Neu5Ac’ or ‘NeuAc’). In some cases the sialic acid component is N-glycolyl-neuraminic acid (Neu5Gc), or a Neu5Ac analogue in which the amine group is replaced by OH (3-deoxy-D-glycero-D-galacto-nonulosonic acid, given the abbreviation ‘KDN’). Gangliosides are defined by a nomenclature system proposed by Svennerholm in which M, D, T and Q refer to mono-, di-, tri- and tetrasialogangliosides, respectively, and the numbers 1, 2, 3, etc. refer to the order of migration of the gangliosides on thin-layer chromatography. For example, the order of migration of monosialogangliosides is GM3>GM2>GM1. To indicate variations within the basic structures, further terms are added, e.g. GM1a, GD1b, etc. Glycosphingolipids having 0, 1, 2, and 3 sialic acid residues linked to the inner galactose unit are termed asialo—(or 0-), a-, b- and c-series gangliosides, respectively, while gangliosides having sialic acid residues linked to the inner N-galactosamine residue are classified as α-series gangliosides. Pathways for the biosynthesis of the 0-, a-, b- and c-series of gangliosides involve sequential activities of sialyltransferases and glycosyltransferases as illustrated eg. in Ledeen et al., 2015 (Ledeen, Robert W., and Gusheng Wu. “The multi-tasked life of GM1 ganglioside, a true factotum of nature.” Trends in biochemical sciences 40.7 (2015): 407-418). Further sialization of each of the series and in different positions in the carbohydrate chain can occur to give an increasingly complex and heterogeneous range of products, such as the α-series gangliosides with sialic acid residue(s) linked to the inner N-acetylgalactosamine residue.

Gangliosides are transferred to the external leaflet of the plasma membrane by a transport system involving vesicle formation. Gangliosides are present and concentrated on cell surfaces, with the two hydrocarbon chains of the ceramide moiety embedded in the plasma membrane and the oligosaccharides located on the extracellular surface, where they present points of recognition for extracellular molecules or surfaces of neighbouring cells. The sialoglycan components of gangliosides extend out from the cell surface, where they can participate in intermolecular interactions. They function by recognizing specific molecules at the cell surface and by regulating the activities of proteins in the plasma membrane. Gangliosides also bind specifically to viruses and to bacterial toxins, such as those from botulinum, tetanus and cholera. For example, the specific cell surface receptor for the cholera toxin is ganglioside GM1 (or GM1a): Neu5Acα2-3(Galβ1-3GalNAcβ1-4)Galβ1-4Glcβ1Cer.

BoNTs possess two independent binding regions in the H_{CC} domain for gangliosides and neuronal protein receptors. BoNT/A, /B, /E, /F and/G have a conserved ganglioside-binding site in the H_{CC} domain composed of a “E(Q) . . . H(K) . . . SXWY . . . G” motif, whereas BoNT/C, /D and/DC display two independent ganglioside-binding sites. (Lam, Kwok-Ho, et al. “Diverse binding modes, same goal: The receptor recognition mechanism of botulinum neurotoxin.” Progress in biophysics and molecular biology 117.2 (2015): 225-231.) Most BoNTs bind only to gangliosides that have an 2,3-linked N-acetylneuraminic acid residue (denoted Sia5) attached to Gal4 of the oligosaccharide core, whereas the corresponding ganglioside-binding pocket on TeNT can also bind to GM1a, a ganglioside lacking the Sia5 sugar residue. It has been shown that introducing a H1241K mutation into a recombinant BoNT/F confers GM1 binding ability (Benson, Marc A., et al. “Unique ganglioside recognition strategies for clostridial neurotoxins.” Journal of Biological Chemistry 286.39 (2011): 34015-34022). BoNT/D has been found to bind GM1a and GD1a (Kroken, Abby R., et al. “Novel ganglioside-mediated entry of botulinum neurotoxin serotype D into neurons.” Journal of Biological Chemistry 286.30 (2011): 26828-26837.)

Combining the data derived from ganglioside-deficient mice and biochemical assays, BoNT/A, E, F and G display a preference for the terminal NAcGal-Gal-NAcNeu moiety being present in GD1a and GT1b, whereas BoNT/B, C, D and TeNT require the disialyl motif found in GD1b, GT1b and GQ1b. Abundant complex polysialo-gangliosides such as GD1a, GD1b and GT1b thus appear essential to specifically accumulate all BoNT serotypes and TeNT on the surface of neuronal cells as the first step of intoxication. (Rummel, Andreas. “Double receptor anchorage of botulinum neurotoxins accounts for their exquisite neurospecificity.” Botulinum Neurotoxins. Springer Berlin Heidelberg, 2012. 61-90.)

The inventors made the hypothesis that advantage could be taken of the differential localization of gangliosides in the body in order to enhance selectivity of clostridial neurotoxins for neurons at specific locations. The inventors have in particular shown that the B subunit of cholera toxin could be used to engineer GM1 binding ability into BoNT/A.

In a first aspect, the present invention provides a hybrid neurotoxin comprising a clostridial light chain and a selective ganglioside binding moiety, wherein the selective ganglioside binding moiety is not a clostridial H_{CC} or H_{C} domain.

The term “neurotoxin” as used herein means any polypeptide that enters a neuron and inhibits neurotransmitter release. This process encompasses the binding of the neurotoxin to a low or high affinity receptor, the internalisation of the neurotoxin, the translocation of the endopeptidase portion of the neurotoxin into the cytoplasm and the enzymatic modification of the neurotoxin substrate. More specifically, the term “neurotoxin” encompasses any polypeptide produced by Clostridium bacteria (clostridial neurotoxins) that enters a neuron and inhibits neurotransmitter release, and such polypeptides produced by recombinant technologies or chemical techniques. It is this dichain form that is the active form of the toxin. The two chains are termed the heavy chain (H-chain), which has a molecular mass of approximately 100 kDa, and the light chain (L-chain), which has a molecular mass of approximately 50 kDa.

Different botulinum neurotoxin (BoNT) serotypes can be distinguished based on inactivation by specific neutralising anti-sera, with such classification by serotype correlating with percentage sequence identity at the amino acid level. BoNT proteins of a given serotype are further divided into different subtypes on the basis of amino acid percentage sequence identity. An example of a BoNT/A amino acid sequence is provided as SEQ ID NO: 1 (UniProt accession number A5HZZ9). An example of a BoNT/B amino acid sequence is provided as SEQ ID NO: 2 (UniProt accession number B1INP5). An example of a BoNT/C amino acid sequence is provided as SEQ ID NO: 3 (UniProt accession number P18640). An example of a BoNT/D amino acid sequence is provided as SEQ ID NO: 4 (UniProt accession number P19321). An example of a BoNT/E amino acid sequence is provided as SEQ ID NO: 5 (UniProt accession number Q00496). An example of a BoNT/F amino acid sequence is provided as SEQ ID NO: 6 (UniProt accession number Q57236). An example of a BoNT/G amino acid sequence is provided as SEQ ID NO: 7 (UniProt accession number Q60393). An example of a TeNT (Tetanus neurotoxin) amino acid sequence is provided as SEQ ID NO: 8 (UniProt accession number P04958).

The term “clostridial light chain” (or “L”) as used herein means a clostridial endopeptidase domain (or non-cytotoxic protease) with a molecular weight of approximately 50 kDa that has the ability to cleave a SNARE protein and thereby disrupt the release of a neurotransmitter from a target cell.

The term “H_{N} domain” as used herein means a functionally distinct region of the neurotoxin heavy chain with a molecular weight of approximately 50 kDa that has the ability to translocate the clostridial light chain into the cytoplasm of a target cell.

The term “LH_{N} domain” as used herein means a neurotoxin that is devoid of the H_{C} domain and consists of an endopeptidase domain (“L” or “light chain”) and the domain responsible for translocation of the endopeptidase into the cytoplasm (H_{N} domain of the heavy chain). A LH_{N} domain comprises an activation site between the L domain and the H_{N} domain. Upon proteolytic cleavage of the activation site, the L domain and the H_{N} domain are joined together by a disulphide bond.

The term “H_{C} domain” as used herein means a functionally distinct region of the neurotoxin heavy chain with a molecular weight of approximately 50 kDa that enables the binding of the neurotoxin to a receptor located on the surface of the target cell. The H_{C} domain consists of two structurally distinct subdomains, the “H_{CN} subdomain” (N-terminal part of the H_{C} domain) and the “H_{CC} subdomain” (C-terminal part of the H_{C} domain), each of which has a molecular weight of approximately 25 kDa.

Exemplary L, H_{N}, H_{CN} and H_{CC} domains are shown in table 1.

TABLE 1
Exemplary L, H_{N}, H_{CN} and H_{CC} domains

| Neurotoxin | Accession Number | SEQ ID NO | L | H_{N} | H_{CN} | H_{CC} |
|---|---|---|---|---|---|---|
| BoNT/A1 | A5HZZ9 | 1 | 1-448 | 449-872 | 873-1094 | 1095-1296 |
| BoNT/B1 | B1INP5 | 2 | 1-441 | 442-859 | 860-1081 | 1082-1291 |
| BoNT/C1 | P18640 | 3 | 1-449 | 450-867 | 868-1095 | 1096-1291 |
| BoNT/D | P19321 | 4 | 1-442 | 443-863 | 864-1082 | 1083-1276 |
| BoNT/E1 | WP_003372387 | 5 | 1-423 | 424-846 | 847-1069 | 1070-1252 |
| BoNT/F1 | Q57236 | 6 | 1-439 | 440-865 | 866-1087 | 1088-1278 |
| BoNT/G | WP_039635782 | 7 | 1-446 | 447-864 | 865-1089 | 1090-1297 |
| TeNT | P04958 | 8 | 1-456 | 457-880 | 881-1111 | 1112-1315 |

The above-identified reference sequences should be considered a guide, as slight variations may occur according to sub-serotypes. By way of example, US 2007/0166332 (hereby incorporated by reference in its entirety) cites slightly different clostridial sequences”.

In one embodiment, the clostridial light chain is from a BoNT type A, type B, type Cl, type D, type E, type F or type G, or a TeNT.

In one embodiment, the clostridial light chain domain comprises a sequence selected from:
amino acid residues 1 to 448 of SEQ ID NO: 1, or a polypeptide sequence having at least 70%, preferably at least 75%, 80%, 85%, 90%, 95% or 99% sequence identity thereto,
amino acid residues 1 to 441 of SEQ ID NO: 2, or a polypeptide sequence having at least 70%, preferably at least 75%, 80%, 85%, 90%, 95% or 99% sequence identity thereto,
amino acid residues 1 to 449 of SEQ ID NO: 3, or a polypeptide sequence having at least 70%, preferably at least 75%, 80%, 85%, 90%, 95% or 99% sequence identity thereto,
amino acid residues 1 to 442 of SEQ ID NO: 4, or a polypeptide sequence having at least 70%, preferably at least 75%, 80%, 85%, 90%, 95% or 99% sequence identity thereto,
amino acid residues 1 to 423 of SEQ ID NO: 5, or a polypeptide sequence having at least 70%, preferably at least 75%, 80%, 85%, 90%, 95% or 99% sequence identity thereto,
amino acid residues 1 to 439 of SEQ ID NO: 6, or a polypeptide sequence having at least 70%, preferably at least 75%, 80%, 85%, 90%, 95% or 99% sequence identity thereto,
amino acid residues 1 to 446 of SEQ ID NO: 7, or a polypeptide sequence having at least 70%, preferably at least 75%, 80%, 85%, 90%, 95% or 99% sequence identity thereto, and
amino acid residues 1 to 456 of SEQ ID NO: 8, or a polypeptide sequence having at least 70%, preferably at least 75%, 80%, 85%, 90%, 95% or 99% sequence identity thereto.

It is understood that a clostridial light chain according to the invention has the ability to cleave a SNARE protein.

In one embodiment, the hybrid neurotoxin comprises a translocation moeity.

The term “translocation moeity” (or “translocation domain”) as used herein means a moiety that has the ability to translocate the clostridial light chain into the cytoplasm of a target cell.

Suitable translocation moieties include bacterial toxin translocation domains such as clostridial H_{N} domains and/or subunit A2 from cholera toxin (CtxA2), cell penetrating peptides, in particular pH sensitive cell penetrating peptides. An example of a pH-sensitive cell penetrating peptide is HBHAc (KKAAPAKKAAAKKAPAKKAAAKK) incorporating a pH-sensitive masking peptide, histidineglutamic acid (HE) (Yeh et al, Mol Pharm 2016 “Selective Intracellular Delivery of Recombinant Arginine Deiminase (ADI) Using pH-Sensitive Cell Penetrating Peptides To Overcome ADI Resistance in Hypoxic Breast Cancer Cells.”).

In a preferred embodiment, the hybrid neurotoxin comprises a translocation moiety which is a clostridial H_{N} domain. In a more preferred embodiment, the hybrid neurotoxin comprises an activation site between the light chain and the clostridial H_{N} domain.

In one embodiment, the clostridial H_{N} domain comprises a sequence selected from:
amino acid residues 449 to 872 of SEQ ID NO: 1, or a polypeptide sequence having at least 70%, preferably at least 75%, 80%, 85%, 90%, 95% or 99% sequence identity thereto,
amino acid residues 442 to 859 of SEQ ID NO: 2, or a polypeptide sequence having at least 70%, preferably at least 75%, 80%, 85%, 90%, 95% or 99% sequence identity thereto,
amino acid residues 450 to 867 of SEQ ID NO: 3, or a polypeptide sequence having at least 70%, preferably at least 75%, 80%, 85%, 90%, 95% or 99% sequence identity thereto,
amino acid residues 442 to 863 of SEQ ID NO: 4, or a polypeptide sequence having at least 70%, preferably at least 75%, 80%, 85%, 90%, 95% or 99% sequence identity thereto,
amino acid residues 423 to 846 of SEQ ID NO: 5, or a polypeptide sequence having at least 70%, preferably at least 75%, 80%, 85%, 90%, 95% or 99% sequence identity thereto,
amino acid residues 440 to 865 of SEQ ID NO: 6, or a polypeptide sequence having at least 70%, preferably at least 75%, 80%, 85%, 90%, 95% or 99% sequence identity thereto,
amino acid residues 447 to 864 of SEQ ID NO: 7, or a polypeptide sequence having at least 70%, preferably at least 75%, 80%, 85%, 90%, 95% or 99% sequence identity thereto, and
amino acid residues 457 to 880 of SEQ ID NO: 8, or a polypeptide sequence having at least 70%, preferably at least 75%, 80%, 85%, 90%, 95% or 99% sequence identity thereto.

It is understood that a clostridial H_{N} domain according to the invention has the ability to translocate the light chain into the cytoplasm of a target cell.

In one embodiment, the clostridial L and H_{N} domains are from the same clostridial serotype.

In one embodiment, the clostridial L and H_{N} domains are from a different clostridial serotype.

In one embodiment, the hybrid neurotoxin comprises a targeting moeity.

The term “targeting moeity” (or “targeting domain”) as used herein means a moiety that has the ability to bind to a receptor on a target cell. Preferably, the targeting moiety has the ability to bind to a protein receptor on a target cell.

Suitable targeting moieties include bacterial toxin targeting domains such as clostridial H_{C} or H_{CC} domains, peptides, antibodies or antibody fragments.

In one embodiment, the hybrid neurotoxin comprises a Targeting Moiety (TM) which binds to a non clostridial receptor. The TM can replace part or all of the H_{C} or H_{CC} domain of the clostridial neurotoxin heavy chain. Hybrid neurotoxins comprising a non clostridial TM may be referred to as “retargeted neurotoxins” (or “targeted secretion inhibitors”, “TSIs”, “TVEMPs” or “TEMs”). Examples of TMs suitable for retargeted neurotoxins are disclosed in WO96/33273, WO98/07864, WO00/10598, WO01/21213, WO01/53336, WO02/07759, WO2005/023309, WO2006/026780, WO2006/099590, WO2006/056093, WO2006/059105, WO2006/059113, WO2007/138339, WO2007/106115, WO2007/106799, WO2009/150469, WO2009/150470, WO2010/055358, WO2010/020811, WO2010/138379, WO2010/138395, WO2010/138382, WO2011/020052, WO2011/020056, WO2011/020114, WO2011/020117, WO2011/20119, WO2012/156743, WO2012/134900, WO2012/134897, WO2012/134904, WO2012/134902, WO2012/135343, WO2012/135448, WO2012/135304, WO2012/134902, WO2014/033441, WO2014/128497, WO2014/053651, WO2015/004464, all of which are herein incorporated by reference.

In one embodiment, the hybrid neurotoxin comprises a clostridial H_{CN} and/or a H_{CC} domain. Preferably, the clostridial H_{CN} and/or H_{CC} domain is from a BoNT type A, type B, type Cl, type D, type E, type F or type G or a TeNT.

In one embodiment, the hybrid neurotoxin comprises a clostridial H_{CN} domain which comprises a sequence selected from:
amino acid residues 873 to 1094 of SEQ ID NO: 1, or a polypeptide sequence having at least 70%, preferably at least 75%, 80%, 85%, 90%, 95% or 99% sequence identity thereto,
amino acid residues 860 to 1081 of SEQ ID NO: 2, or a polypeptide sequence having at least 70%, preferably at least 75%, 80%, 85%, 90%, 95% or 99% sequence identity thereto,
amino acid residues 868 to 1095 of SEQ ID NO: 3, or a polypeptide sequence having at least 70%, preferably at least 75%, 80%, 85%, 90%, 95% or 99% sequence identity thereto,
amino acid residues 864 to 1082 of SEQ ID NO: 4, or a polypeptide sequence having at least 70%, preferably at least 75%, 80%, 85%, 90%, 95% or 99% sequence identity thereto,
amino acid residues 847 to 1069 of SEQ ID NO: 5, or a polypeptide sequence having at least 70%, preferably at least 75%, 80%, 85%, 90%, 95% or 99% sequence identity thereto,
amino acid residues 866 to 1087 of SEQ ID NO: 6, or a polypeptide sequence having at least 70%, preferably at least 75%, 80%, 85%, 90%, 95% or 99% sequence identity thereto,
amino acid residues 865 to 1089 of SEQ ID NO: 7, or a polypeptide sequence having at least 70%, preferably at least 75%, 80%, 85%, 90%, 95% or 99% sequence identity thereto, or
amino acid residues 881 to 1111 of SEQ ID NO: 8, or a polypeptide sequence having at least 70%, preferably at least 75%, 80%, 85%, 90%, 95% or 99% sequence identity thereto.

In one embodiment, the hybrid neurotoxin comprises a clostridial H_{CC} domain which comprises a sequence selected from:
amino acid residues 1095 to 1296 of SEQ ID NO: 1, or a polypeptide sequence having at least 70%, preferably at least 75%, 80%, 85%, 90%, 95% or 99% sequence identity thereto,
amino acid residues 1082 to 1291 of SEQ ID NO: 2, or a polypeptide sequence having at least 70%, preferably at least 75%, 80%, 85%, 90%, 95% or 99% sequence identity thereto,
amino acid residues 1096 to 1291 of SEQ ID NO: 3, or a polypeptide sequence having at least 70%, preferably at least 75%, 80%, 85%, 90%, 95% or 99% sequence identity thereto,
amino acid residues 1083 to 1276 of SEQ ID NO: 4, or a polypeptide sequence having at least 70%, preferably at least 75%, 80%, 85%, 90%, 95% or 99% sequence identity thereto,
amino acid residues 1070 to 1252 of SEQ ID NO: 5, or a polypeptide sequence having at least 70%, preferably at least 75%, 80%, 85%, 90%, 95% or 99% sequence identity thereto,
amino acid residues 1088 to 1278 of SEQ ID NO: 6, or a polypeptide sequence having at least 70%, preferably at least 75%, 80%, 85%, 90%, 95% or 99% sequence identity thereto,
amino acid residues 1090 to 1297 of SEQ ID NO: 7, or a polypeptide sequence having at least 70%, preferably at least 75%, 80%, 85%, 90%, 95% or 99% sequence identity thereto, or
amino acid residues 1112 to 1315 of SEQ ID NO: 8, or a polypeptide sequence having at least 70%, preferably at least 75%, 80%, 85%, 90%, 95% or 99% sequence identity thereto.

It is understood that a H_{CC} according to the invention is capable of binding to a clostridial neurotoxin protein receptor.

In one embodiment, the clostridial H_{CN} and/or H_{CC} domain are from the same clostridial serotype as the light chain.

In one embodiment, the clostridial H_{CN} and/or H_{CC} domain are from a different clostridial serotype as the light chain.

In one embodiment, the hybrid neurotoxin comprises a clostridial H_{CN} domain and a clostridial H_{CC} domain. Suitably, the clostridial H_{CN} and H_{CC} domains can be from the same serotype. Suitably, the clostridial H_{CN} and H_{CC} domains can be from a different serotype. In one embodiment, the clostridial light chain, H_{CN} and H_{CC} domain are from the same serotype. In one embodiment, the clostridial light chain and H_{CN} are from the same serotype and the H_{CC} domain is from a different serotype.

In one embodiment, the hybrid neurotoxin comprises a clostridial H_{N} domain, a clostridial H_{CN} domain and a clostridial H_{CC} domain. Suitably, the clostridial H_{N}, H_{CN} and H_{CC} domains can be from the same serotype. Suitably, the clostridial H_{N}, H_{CN} and H_{CC} domains can be from different serotypes. In one embodiment, the clostridial light chain, H_{N}, H_{CN} and H_{CC} domains are from the same serotype. In one embodiment, the clostridial light chain, H_{N} and H_{CN} domains are from the same serotype and the H_{CC} domain is from a different serotype. In one embodiment, the clostridial light chain and H_{N} domain are from the same serotype and the H_{CN} and H_{CC} domains are from a different serotype.

In one embodiment, when the hybrid neurotoxin comprises a clostridial H_{CC} domain, the clostridial H_{CC} domain has an ability to bind to gangliosides which is reduced or abolished as compared to a native clostridial H_{CC} domain. This may be achieved for example by introducing mutations into the ganglioside binding motif of the H_{CC} domain.

The “percent sequence identity” between two or more nucleic acid or amino acid sequences is a function of the number of identical nucleotides/amino acids at identical positions shared by the aligned sequences. Thus, % identity may be calculated as the number of identical nucleotides/amino acids at each position in an alignment divided by the total number of nucleotides/amino acids in the aligned sequence, multiplied by 100. Calculations of % sequence identity may also take into account the number of gaps, and the length of each gap that needs to be introduced to optimize alignment of two or more sequences. Sequence comparisons and the determination of percent identity between two or more sequences can be carried out using specific mathematical algorithms, which will be familiar to a skilled person, for example a global alignment mathematical algorithm (such as described by Needleman and Wunsch, J. Mol. Biol. 48(3), 443-453, 1972).

The light chain, H_{N}, H_{CN} and H_{CC} domains can be from a mosaic neurotoxin. The term “mosaic neurotoxin” as used in this context refers to a naturally occurring clostridial neurotoxin that comprises at least one functional domain from another type of clostridial neurotoxins (e.g. a clostridial neurotoxin of a different serotype), the clostridial neurotoxin not usually comprising the at least one functional domain. Examples of mosaic neurotoxins are naturally occurring BoNT/DC and BoNT/CD. BoNT/DC comprises the L chain and H_{N} domain of serotype D and the H_{C} domain of serotype C, whereas BoNT/CD consists of the L chain and H_{N} domain of serotype C and the H_{C} domain of serotype D.

The light chain, H_{N}, H_{CN} and H_{CC} domains can be from a modified neurotoxin and derivatives thereof, including but not limited to those described below. A modified neurotoxin or derivative may contain one or more amino acids that has been modified as compared to the native (unmodified) form of the neurotoxin, or may contain one or more inserted amino acids that are not present in the native (unmodified) form of the toxin. By way of example, a modified clostridial neurotoxin may have modified amino acid sequences in one or more domains relative to the native (unmodified) clostridial neurotoxin sequence. Such modifications may modify functional aspects of the neurotoxin, for example biological activity or persistence.

A modified neurotoxin retains at least one of the functions of a neurotoxin, selected from the ability to bind to a low or high affinity neurotoxin receptor on a target cell, to translocate the endopeptidase portion of the neurotoxin (light chain) into the cell cytoplasm and to cleave a SNARE protein. Preferably, a modified neurotoxin retains at least two of these functions. More preferably a modified neurotoxin retains these three functions.

A modified neurotoxin may have one or more modifications in the amino acid sequence of the heavy chain (such as a modified H_{C} domain), wherein the modified heavy chain binds to target nerve cells with a higher or lower affinity than the native (unmodified) neurotoxin. Such modifications in the H_{C} domain can include modifying residues in the ganglioside binding site of the H_{C} domain or in the protein (SV2 or synaptotagmin) binding site that alter binding to the ganglioside receptor and/or the protein receptor of the target nerve cell. Examples of such modified neurotoxins are described in WO 2006/027207 and WO 2006/114308, both of which are hereby incorporated by reference in their entirety.

A modified neurotoxin may have one or more modifications in the amino acid sequence of the light chain, for example modifications in the substrate binding or catalytic domain which may alter or modify the SNARE protein specificity of the modified LC. Examples of such modified neurotoxins are described in WO 2010/120766 and US 2011/0318385, both of which are hereby incorporated by reference in their entirety.

A modified neurotoxin may comprise one or more modifications that increases or decreases the biological activity and/or the biological persistence of the modified neurotoxin. For example, a modified neurotoxin may comprise a leucine- or tyrosine-based motif, wherein the motif increases or decreases the biological activity and/or the biological persistence of the modified neurotoxin. Suitable leucine-based motifs include xDxxxLL, xExxxLL, xExxxlL, and xExxxLM (wherein x is any amino acid). Suitable tyrosine-based motifs include Y-x-x-Hy (wherein Hy is a hydrophobic amino acid). Examples of modified neurotoxins comprising leucine- and tyrosine-based motifs are described in WO 2002/08268, which is hereby incorporated by reference in its entirety.

The term “selective ganglioside binding moiety” as used herein means a moiety that binds to one type of ganglioside with a higher affinity than to other gangliosides. The binding affinity can be quantified by determining the equilibrium dissociation constant or K_{d} between the ganglioside and the binding moiety (the lower the K_{d} the higher the affinity). Methods for determining binding affinity for a ganglioside are well known in the art, and include for example surface plasmon resonance (SPR), as described for example in Kuziemko, Geoffrey M. et al. “Cholera toxin binding affinity and specificity for gangliosides determined by surface plasmon resonance.” Biochemistry 35.20 (1996): 6375-6384, or in MacKenzie, C. Roger, et al. “Quantitative analysis of bacterial toxin affinity and specificity for glycolipid receptors by surface plasmon resonance.” Journal of Biological Chemistry 272.9 (1997): 5533-5538. In particular, Kuziemko et al., 1996 (cited above) found by using SPR that the cholera toxin preferably binds to gangliosides in the following sequence: GM1>GM2>GD1A>GM3>GT1B>GD1B>asialo-GM1, and that the measured binding affinity (K_{d}) of cholera toxin for the ganglioside sequence ranges from 4.61×10⁻¹² M for GM1 to 1.88×10⁻¹⁰ M for asialo GM1. MacKenzie et al., 1997 (cited above) determined by surface plasmon resonance (SPR) using a liposome capture method that CtxB binds to GM1 and GD1b with an affinity of respectively 7.3×10⁻¹⁰ M and 8×10⁻⁹ M, that E. coli heat labile enterotoxin (LT) binds to GM1 and GD1b with an affinity of respectively 5.7×10⁻¹⁰ M and 3.0×10⁻⁹ M, and that tetanus toxin C fragment binds to GD1b and GT1b with an affinity of respectively 1.5×10⁻′M and 1.7×10⁻⁷ M.

The binding affinity for a ganglioside can also be determined by using a competitive ELISA assay, as described for example in Sinclair, Haydn R., et al. “Sialyloligosaccharides inhibit cholera toxin binding to the GM1 receptor.” Carbohydrate research 343.15 (2008): 2589-2594. Another method for determining the binding affinity for a ganglioside is based on the use of a radiolabelled ligand (for example ¹²⁵I-labeled), as described for example in Nishiki, Tei-ichi, et al. “The high-affinity binding of Clostridium botulinum type B neurotoxin to synaptotagmin II associated with gangliosides GT1b/GD1a.” FEBS letters 378.3 (1996): 253-257. Yet another method for determining ganglioside binding affinity is the use of isothermal titration calorimetry as described in Turnbull, W. Bruce, et al. “Dissecting the cholera toxin-ganglioside GM1 interaction by isothermal titration calorimetry.” Journal of the American Chemical Society 126.4 (2004): 1047-1054.

In one embodiment, the K_{d} between the selective ganglioside binding moiety and the ganglioside is lower than about 10⁻⁹ M, preferably lower than about 10⁻¹⁰ M, more preferably lower than about 10⁻¹¹ M, more preferably lower than about 5×10⁻¹² M.

Suitable ganglioside binding moieties (GBM) include bacterial toxin GBMs (other than clostridial H_{C} or H_{CC} domains), peptides, proteins or protein fragments, antibodies or antibody fragments.

In one embodiment, the GBM is a peptide. Examples of peptides suitable for use as a GBM include Alzheimer's β-amyloid peptide (Aβ) which binds to GM1, Parkinson's disease associated protein α-synuclein which binds to GM3, and chimeric peptides such as α-synuclein/Aβ described in Yahi and Fantini 2014, which binds to GM1 and GM3 (Yahi, Nouara, and Jacques Fantini. “Deciphering the glycolipid code of Alzheimer's and Parkinson's amyloid proteins allowed the creation of a universal ganglioside-binding Peptide.” PloS one 9.8 (2014): e104751).

In one embodiment, the GBM is a protein or protein fragment. Examples of proteins suitable for use as a GBM include growth factor receptors, such as the epidermal growth factor receptor (EGFR) which binds to GM3, GM1, GM2, GM4, GD3, GD1a and GT1b, and the vascular endothelial growth factor receptor (VEGFR) which binds to GM3, GD1a and GT1b (Krengel, Ute, and Paula A. Bousquet. “Molecular recognition of gangliosides and their potential for cancer immunotherapies.” Frontiers in Immunology, 2014, vol 5, article 325).

In a preferred embodiment, the GBM is from a bacterial toxin, for example the GBM is selected from Cholera toxin B subunit (CtxB) and E. coli heat labile enterotoxin (LT).

GM1 (or GM1a) is the only known receptor for the Cholera toxin B subunit (CtxB). Therefore, by engineering the B subunit of the cholera toxin into a clostridial neurotoxin, or a fragment thereof, it is possible to selectively target it to GM1 containing neurons. GM1 is also a receptor for the heat-labile enterotoxin of Escherichia coli. (Zoeteweij, J. Paul, et al. “GM1 binding-deficient exotoxin is a potent noninflammatory broad spectrum intradermal immunoadjuvant.” The Journal of Immunology 177.2 (2006): 1197-1207).

It is also hypothesized that a hybrid neurotoxin according to the invention in which the GBM is from a bacterial toxin is particularly suitable for topical delivery of the hybrid neurotoxin. Indeed, it has been shown also that bacterial exotoxins can be safely used topically on the skin in humans (Zoeteweij, J. Paul, et al. “GM1 binding-deficient exotoxin is a potent noninflammatory broad spectrum intradermal immunoadjuvant.” The Journal of Immunology 177.2 (2006): 1197-1207).

In one embodiment, the ganglioside is GM1, and the GBM is selected from CtxB and E. coli heat labile enterotoxin (LT).

Cholera toxin (CT) is secreted by the gram-negative bacterium Vibrio cholerae. CT belongs to the larger family of AB toxins which have an enzymatically active A-domain responsible for inducing toxicity, and a cell binding B-domain responsible for cell entry. CT belongs to the AB₅ subfamily which is comprised of six polypeptides, a single A-subunit and a homopentameric B-subunit that self-assemble to form the holotoxin prior to secretion. Other AB₅ toxins include the heat labile enterotoxins, the shiga toxin, the shiga-like toxins and the pertussis toxin. The CT A- and B-subunits (CtxA and CtxB respectively) are non-covalently linked. The 27 kDa A-subunit contains a serine-protease cleavage site located between residues 192 and 195 that allows for cleavage of the A-subunit into two polypeptides: the A2-chain and A1-chain. A disulfide bond between residues 187 and 199 bridges these chains together. The A1 chain is responsible for the enzymatic activity of CT. Five 11.5 kDa B-subunits assemble non-covalently to form a homopentamer that binds to the ganglioside GM1 on the plasma membrane. The B5-subunit-GM1 complex carries the A-subunit into the endoplasmic reticulum. Following retro-translocation, the A1-chain enters the cytosol as an active ADP-ribosyltransferase that modifies the heterotrimeric G protein, Gsa. Modification of this G protein leads to the constitutive activation of adenylate cyclase and the rapid production of cAMP. In intestinal cells, this induces intestinal chloride secretion, which is accompanied by a massive movement of water and the diarrhea that is the hallmark of cholera. (Wernick, Naomi L B, et al. “Cholera toxin: an intracellular journey into the cytosol by way of the endoplasmic reticulum.” Toxins 2.3 (2010): 310-325.)

An example of a Cholera toxin B subunit amino acid sequence is provided as SEQ ID NO: 9 (UniProtKB accession number P01556) which consists of a signal peptide (amino acid residues 1 to 21 of SEQ ID NO:9) and the B subunit (amino acid residues 22 to 124 of SEQ ID NO:9).

An example of a Cholera toxin A subunit amino acid sequence is provided as SEQ ID NO: 10 (UniProtKB accession number P01555), which consists of a signal peptide (amino acid residues 1 to 18 of SEQ ID NO:10), the A1 domain (amino acid residues 19 to 212 of SEQ ID NO:10) and the A2 domain (amino acid residues 213 to 258 of SEQ ID NO:10).

It has been shown that a monomeric B subunit is sufficient to bind to cells and complete the intoxication pathway (Jobling, Michael G., et al. “A single native ganglioside GM1-binding site is sufficient for cholera toxin to bind to cells and complete the intoxication pathway.” MBio 3.6 (2012): e00401-12).

The inventors have shown that the addition of a CtxB domain to an endonegative BoNT/A1 (BoNT/A1(0)) confers BoNT/A1 with the ability to bind to GM1, a ganglioside which is not a natural receptor for BoNT/A1.

Without wishing to be bound by theory, it is hypothesized that the CtxB subunit will result in increased potency due to the fact that Cholera toxin has a greater binding affinity for GM1 than BoNTs have for their corresponding gangliosides. It has for example been shown that the affinity of BoNT/B to the complex synaptotagmin associated with GT1b/GD1a (dual receptor model) is in the nM range (“high affinity 0.4 nM, low affinity 4.1 nM”) (Nishiki et al, FEBS Letters 1996), which is 1000 fold more than the pM affinity reported in Kuzimeko et al, Biochemistry 1996 between Ctx-B and GM1.

A CtxB domain according to the invention preferably comprises amino acid residues 22 to 124 of SEQ ID NO: 9, or a polypeptide sequence having at least 70%, preferably at least 75%, 80%, 85%, 90%, 95% or 99% sequence identity thereto. It is understood that a CtxB domain according to the invention is capable of binding to GM1.

In a preferred embodiment, the selective ganglioside binding moiety comprises one or more Cholera toxin B subunits (CtxB).

In one embodiment, the light chain is covalently bound to said one or more Cholera toxin B subunits (CtxB).

In one embodiment, the selective ganglioside binding moiety comprises one CtxB. In one embodiment, the selective ganglioside binding moiety comprises two CtxB. In one embodiment, the selective ganglioside binding moiety comprises three CtxB. In one embodiment, the selective ganglioside binding moiety comprises four CtxB. In one embodiment, the selective ganglioside binding moiety comprises five CtxB.

In one embodiment, the selective ganglioside binding moiety comprises one or more CtxB which are C-terminal to the clostridial light chain. In one embodiment, the selective ganglioside binding moiety comprises one or more CtxB which are N-terminal to the clostridial light chain. In one embodiment, the selective ganglioside binding moiety consists of one CtxB which is C-terminal to the clostridial light chain. In one embodiment, the selective ganglioside binding moiety consists of one CtxB which is N-terminal to the clostridial light chain. In one embodiment, the selective ganglioside binding moiety consists of two CtxB which are C-terminal to the clostridial light chain. In one embodiment, the selective ganglioside binding moiety consists of two CtxB which are N-terminal to the clostridial light chain. In one embodiment, the selective ganglioside binding moiety consists of three CtxB which are C-terminal to the clostridial light chain. In one embodiment, the selective ganglioside binding moiety consists of three CtxB which are N-terminal to the clostridial light chain. In one embodiment, the selective ganglioside binding moiety consists of four CtxB which are C-terminal to the clostridial light chain. In one embodiment, the selective ganglioside binding moiety consists of four CtxB which are N-terminal to the clostridial light chain. In one embodiment, the selective ganglioside binding moiety consists of five CtxB which are C-terminal to the clostridial light chain. In one embodiment, the selective ganglioside binding moiety consists of five CtxB which are N-terminal to the clostridial light chain.

In one embodiment, the hybrid neurotoxin comprises a clostridial H_{N} domain and the selective ganglioside binding moiety comprises one or more CtxB which are C-terminal to the clostridial H_{N} domain. In another embodiment, the one or more CtxB are N-terminal to the clostridial H_{N} domain. In one embodiment, the selective ganglioside binding moiety consists of one CtxB which is C-terminal to the clostridial H_{N}. In one embodiment, the selective ganglioside binding moiety consists of one CtxB which is N-terminal to the clostridial H_{N}. In one embodiment, the selective ganglioside binding moiety consists of two CtxB which are C-terminal to the clostridial H_{N}. In one embodiment, the selective ganglioside binding moiety consists of two CtxB which are N-terminal to the clostridial H_{N}. In one embodiment, the selective ganglioside binding moiety consists of three CtxB which are C-terminal to the clostridial H_{N}. In one embodiment, the selective ganglioside binding moiety consists of three CtxB which are N-terminal to the clostridial H_{N}. In one embodiment, the selective ganglioside binding moiety consists of four CtxB which are C-terminal to the clostridial H_{N}. In one embodiment, the selective ganglioside binding moiety consists of four CtxB which are N-terminal to the clostridial H_{N}. In one embodiment, the selective ganglioside binding moiety consists of five CtxB which are C-terminal to the clostridial H_{N}. In one embodiment, the selective ganglioside binding moiety consists of five CtxB which are N-terminal to the clostridial H_{N}.

In one embodiment, the selective ganglioside binding moiety comprises one or more Cholera toxin B subunits (CtxB) and the hybrid neurotoxin further comprises a Cholera toxin A2 subunit (CtxA2). Preferably, the CtxA2 is covalently bound to the clostridial light chain. Preferably still, the CtxA2 is covalently bound to the clostridial light chain and the CtxB forms a non covalent link with the clostridial light chain. It is in particularly considered the Cholera toxin A2 subunit (CtxA2) could act as a tether to form a non-covalent link with the B subunit pentamer (CtxB₅) which will bind to the ganglioside on a target cell and internalise the clostridial light chain into the cell.

Without wishing to be bound by theory, it is hypothesized that such embodiments in which the CtxB binds to hybrid neurotoxin through a non-covalent link (to the CtxA2 subunit) allows for a pentameric arrangement of the CtxB subunit (CtxB₅), and thereby results in an increased binding affinity of the hybrid neurotoxin for GM1.

A CtxA2 domain according to the invention preferably comprises amino acid residues 213 to 258 of SEQ ID NO: 10, or a polypeptide sequence having at least 70%, preferably at least 75%, 80%, 85%, 90%, 95% or 99% sequence identity thereto. It is understood that a CtxA2 domain according to the invention is capable of binding to a CtxB domain. Preferably, a CtxA2 domain comprises residues 255 to 258 (KDEL) of SEQ ID NO: 10.

The CtxA2 domain can be C-terminal or N-terminal to the clostridial light chain.

In one embodiment, the selective ganglioside binding moiety comprises one or more Cholera toxin B subunits (CtxB) which are non covalently linked to the clostridial light chain, and the hybrid neurotoxin further comprises a Cholera toxin A2 subunit (CtxA2) and a H_{N} domain which are covalently bound to the clostridial light chain. In one embodiment, the CtxA2 domain is N-terminal to the clostridial H_{N} domain and is preferably located between the activation site and the H_{N} domain (“central presentation”). In one embodiment, the CtxA2 domain is C-terminal to the clostridial H_{N} domain, and when the hybrid neurotoxin includes a H_{CN} and/or a H_{CC} domain, the ganglioside binding moiety may be located C-terminal or N-terminal to the H_{CN} or H_{CC} domain. The hybrid neurotoxin may comprise a linker between the CtxA2 domain and the L, H_{N}, H_{CN} and/or H_{CC} domain.

In one embodiment, the clostridial light chain is covalently linked to the selective ganglioside binding moiety. The selective ganglioside binding moiety can be C-terminal or N-terminal to the clostridial light chain.

In one embodiment, the hybrid neurotoxin comprises a clostridial H_{N} domain and the clostridial H_{N} domain is covalently linked to the selective ganglioside binding moiety. The selective ganglioside binding moiety can be C-terminal or N-terminal to the clostridial H_{N} domain. When the selective ganglioside binding moiety is N-terminal to the clostridial H_{N} domain, it is preferably located between the activation site and the H_{N} domain (“central presentation”).

When the selective ganglioside binding moiety is C-terminal to the clostridial H_{N} domain and when the hybrid neurotoxin further comprises a H_{CN} domain and/or a H_{CC} domain, the ganglioside binding moiety may be located C-terminal or N-terminal to the H_{CN} or H_{CC} domain.

The hybrid neurotoxin may comprise a linker between the ganglioside binding domain and the L, H_{N}, H_{CN} and/or H_{CC} domain. Without wishing to be bound by theory, it is hypothesized that the presence of a linker can enhance the stability of the hybrid neurotoxin and/or the availability of the ganglioside binding moiety for its target ganglioside, and/or increase expression.

Examples of suitable linkers include GS linkers of varying length, eg GS5, GS10, GS15, GS18 and GS20, N10, HX27, (EAAAK)₃ and A(EAAAK)₄ALEA(EAAAK)₄A. Further examples are provided in the literature, for example in Chen, Xiaoying, et al. “Fusion protein linkers: property, design and functionality.” Advanced drug delivery reviews 65.10 (2013): 1357-1369, herein incorporated by reference.

Examples of structural arrangement of hybrid neurotoxins according to the invention are shown below (GBM: ganglioside binding moiety; TD: translocation domain; BD: protein receptor binding domain; L, H_{N}, H_{CN}, H_{CC}: clostridial domains as defined herein; AS: activation site; from left to right: C-terminal to N-terminal):
L-GBM
GBM-L
L-AS-GBM
GBM-AS-L
L-AS-GBM-TD
L-AS-TD-GBM
GBM-TD-AS-L
TD-GBM-AS-L
GBM-L-AS-TD
L-AS-GBM-TD-BD
L-AS-BD-TD-GBM
GBM-TD-BD-AS-L
BD-TD-GBM-AS-L
L-AS-GBM-H_{N}
L-AS-H_{N}-GBM
GBM-H_{N}-AS-L
H_{N}-GBM-AS-L
GBM-L-AS-H_{N}
L-AS-GBM-H_{N}-H_{CN}
L-AS-GBM-H_{N}-H_{CN}-H_{CC}
L-AS-H_{N}-GBM
L-AS-H_{N}-H_{CN}-GBM
L-AS-H_{N}-H_{CN}-H_{CC}-GBM
L-AS-GBM-linker-H_{N}
L-AS-GBM-linker-H_{N}-H_{CN}
L-AS-GBM-linker-H_{N}-H_{CN}-H_{CC}
L-AS-H_{N}-linker-GBM
L-AS-H_{N}-H_{CN}-linker-GBM
L-AS-H_{N}-H_{CN}-H_{CC}-linker-GBM
GBM-L-AS-H_{N}-H_{CN}-H_{CC}
L-AS-linker-GBM-H_{N}
L-AS-linker-GBM-H_{N}-H_{CN}
L-AS-linker-GBM-H_{N}-H_{CN}-H_{CC}
L-AS-linker-GBM-linker-H_{N}
L-AS-linker-GBM-linker-H_{N}-H_{CN}
L-AS-linker-GBM-linker-H_{N}-H_{CN}-H_{CC}

In one embodiment, the hybrid neurotoxin of the invention comprises a H_{N} domain and is in a dichain form and comprises a di-sulfide bond between the L domain and the H_{N} domain.

Preferably, the structural arrangement of the hybrid neurotoxin is such that the GBM has a free N-terminal or C-terminal end. In embodiments comprising an activation site (AS) allowing for conversion of the hybrid neurotoxin into a dichain form, the structural arrangement of the hybrid neurotoxin is preferably such that the GBM has a free N-terminal or C-terminal end after conversion into the dichain form.

In embodiments comprising a protein receptor binding domain (BD), for example a H_{C} or a H_{CC}, the structural arrangement of the hybrid neurotoxin is preferably such that the BD has a free N-terminal or C-terminal end, and more preferably that both the GBM and the BD have free N-terminal or C-terminal end. In embodiments comprising an activation site (AS) allowing for conversion of the hybrid neurotoxin into a dichain form, the structural arrangement of the hybrid neurotoxin is preferably such that the BD has a free N-terminal or C-terminal end after conversion into the dichain form, and more preferably that both the GBM and the BD have free N-terminal or C-terminal end after conversion into the dichain form.

The hybrid neurotoxins of the present invention can be produced using recombinant technologies. Thus, in one embodiment, a hybrid neurotoxin according to the invention is a recombinant hybrid neurotoxin.

In another aspect, the invention provides a nucleotide sequence encoding a hybrid neurotoxin according to the invention, for example a DNA or RNA sequence. In a preferred embodiment, the nucleotide sequence is a DNA sequence.

The nucleic acid molecules of the invention may be made using any suitable process known in the art. Thus, the nucleic acid molecules may be made using chemical synthesis techniques. Alternatively, the nucleic acid molecules of the invention may be made using molecular biology techniques.

The DNA sequence of the present invention is preferably designed in silico, and then synthesised by conventional DNA synthesis techniques.

The above-mentioned nucleic acid sequence information is optionally modified for codon-biasing according to the ultimate host cell (e.g. E. coli) expression system that is to be employed.

In another aspect, the invention provides a vector comprising a nucleotide sequence according to the invention. In one embodiment, the nucleic acid sequence is prepared as part of a DNA vector comprising a promoter and terminator. In a preferred embodiment, the vector has a promoter selected from Tac, AraBAD, T7-Lac, or T5-Lac.

A vector may be suitable for in vitro and/or in vivo expression of the above-mentioned nucleic acid sequence. The vector can be a vector for transient and/or stable gene expression. The vector may additionally comprise regulatory elements and/or selection markers. The vector may be of viral origin, of phage origin, or of bacterial origin. For example, the expression vector may be a pET, pJ401, pGEX vector or a derivative thereof.

In another aspect, the invention provides a cell comprising a nucleotide sequence or a vector according to the invention. Suitable cell types include prokaryotic cells, for example E. coli, and eukaryotic cells, such as yeast cells, mammalian cells, insect cells . . . Preferably, the cell is E. coli.

The hybrid neurotoxins of the invention are particularly suitable for use in therapy.

The Guillain-Barré syndrome is an acute inflammatory disorder which affects the peripheral nervous system and is caused by the binding of antibodies produced by the immune system to gangliosides. Based on findings from the clinical subtypes of the Guillain-Barré syndrome, gangliosides GM1a, GM1b, GD1a, GalNAc-GD1a have been linked to the neuromuscular junction of the limbs, and gangliosides GT1a, GQ1b have been linked to head-and-neck neuromuscular junctions (Van Den Berg, Bianca, et al. “Guillain-Barré syndrome: pathogenesis, diagnosis, treatment and prognosis.” Nature reviews. Neurology 10.8 (2014): 469; Willison, Hugh J., and Jaap J. Plomp. “Anti-ganglioside Antibodies and the Presynaptic Motor Nerve Terminal.” Annals of the New York Academy of Sciences 1132.1 (2008): 114-123). GM1 has also been shown to be abundant in the parotid glands (salivary glands) (Nowroozi, Nakisa, et al. “HIGH LEVELS OF GM 1-GANGLIOSIDE AND GM 1-GANGLIOSIDE 13-GALACTOSIDASE IN THE PAROTID GLAND: A New Model for Secretory Mechanisms of the Parotid Gland.” Otolaryngologic Clinics of North America 32.5 (1999): 779-791).

GM1 and GM2 concentrations in lipid rafts from the frontal and temporal cortex were reported to be higher in Alzheimer's disease (AD) patients. GM1 clustering was demonstrated in dorsal root ganglion neurons (sensory neurons). (Aureli, Massimo, et al. “GM1 ganglioside: past studies and future potential.” Molecular neurobiology 53.3 (2016): 1824-1842.)

Gangliosides NeuAc GM3, NeuGc GM3, GM2, GM1, GD3 and GD2 have been shown to be expressed in human tumour cells (Krengel, Ute, and Paula A. Bousquet. “Molecular recognition of gangliosides and their potential for cancer immunotherapies.” Frontiers in Immunology, July 2014, vol. 5, article 325).

In one embodiment, the selective ganglioside binding moiety binds to one or more gangliosides selected from GM1a, GM1b, GD1a and GalNAc-GD1a. It is believed that such embodiments are particularly suitable for treating limb disorders such as upper limb spasticity, lower limb spasticity, focal hand dystonia, limb muscle strain, repetitive strain injury (RSI), cumulative trauma disorder or occupational overuse syndrome. Indeed, without wishing to be bound by theory, it is hypothesized that targeting the hybrid neurotoxin to gangliosides found at the neuromuscular junction of the limbs allows to increase selectivity for limb neuromuscular junctions and to avoid side effects due to off-target effects.

In one embodiment, the selective ganglioside binding moiety binds to one or more gangliosides selected from GT1a and GQ1b. It is believed that such embodiments are particularly suitable for treating head and neck disorders such as cervical dystonia, blepharospasm, migraine, myofascial pain, strabismus, hemifacial spasm, eyelid disorder, spasmodic dysphonia, laryngeal dystonia, oromandibular dysphonia, lingual dystonia, bruxism and dysphagia. Indeed, without wishing to be bound by theory, it is hypothesized that targeting the hybrid neurotoxin to gangliosides found at the head and neck neuromuscular junctions allows to increase selectivity for head and neck neuromuscular junctions and to avoid side effects due to off-target effects.

In one embodiment, the selective ganglioside binding moiety binds to GM1. It is believed this embodiment is particularly suitable for treating sialorrhea (excessive salivation, drooling). It is also hypothesized that this embodiment could be suitable for treating patients suffering from Alzheimer's disease or other neurological disorders.

In one embodiment, the selective ganglioside binding moiety binds to one or more gangliosides selected from NeuAc GM3, NeuGc GM3, GM2, GM1, GD3 and GD2. It is believed that such embodiments are particularly suitable for treating cancer.

In another aspect, the invention provides a pharmaceutical composition comprising a hybrid neurotoxin according to the invention. Preferably, the pharmaceutical composition comprises a hybrid neurotoxin together with at least one component selected from a pharmaceutically acceptable carrier, excipient, adjuvant, propellant and/or salt.

In another aspect, the invention provides a hybrid neurotoxin or pharmaceutical composition according to the invention for use in therapy.

A hybrid neurotoxin or pharmaceutical composition according to the invention is suitable for use in treating a condition associated with unwanted neuronal activity, for example a condition selected from the group consisting of spasmodic dysphonia, spasmodic torticollis, laryngeal dystonia, oromandibular dysphonia, lingual dystonia, cervical dystonia, focal hand dystonia, blepharospasm, strabismus, hemifacial spasm, eyelid disorder, cerebral palsy, focal spasticity and other voice disorders, spasmodic colitis, neurogenic bladder, anismus, limb spasticity, tics, tremors, bruxism, anal fissure, achalasia, dysphagia and other muscle tone disorders and other disorders characterized by involuntary movements of muscle groups, lacrimation, hyperhidrosis, excessive salivation, excessive gastrointestinal secretions, secretory disorders, pain from muscle spasms, headache pain, migraine and dermatological conditions.

In another aspect, the invention provides a non-therapeutic use of a hybrid neurotoxin or pharmaceutical composition according to the invention for treating an aesthetic or cosmetic condition. According to this aspect of the invention, the subject to be treated for an aesthetic or cosmetic condition is preferably not suffering from any of the pathological disorders or conditions that are described herein. More preferably, said subject is a healthy subject (i.e. not suffering from any pathological disease or condition).

In another aspect, the invention provides a hybrid neurotoxin comprising a clostridial light chain and a selective ganglioside binding moiety, wherein the selective ganglioside binding moiety is not a clostridial H_{CC} or H_{C} domain, and wherein said selective ganglioside binding moiety binds to one or more gangliosides selected from GM1a, GM1b, GD1a and GalNAc-GD1a, for use in treating a limb disorder associated with unwanted neuronal activity. In one embodiment, the limb disorder is selected from upper limb spasticity, lower limb spasticity and focal hand dystonia. In a preferred embodiment, the ganglioside is GM1a.

In another aspect, the invention provides a hybrid neurotoxin comprising a clostridial light chain and a selective ganglioside binding moiety, wherein the selective ganglioside binding moiety is not a clostridial H_{CC} or H_{C} domain, and wherein said selective ganglioside binding moiety binds to one or more gangliosides selected from GT1a and GQ1b, for use in treating a head or neck disorder associated with unwanted neuronal activity. In one embodiment, the head or neck disorder is selected from cervical dystonia, blepharospasm, migraine, myofascial pain, strabismus, hemifacial spasm, eyelid disorder, spasmodic dysphonia, laryngeal dystonia, oromandibular dysphonia, lingual dystonia, bruxism and dysphagia.

In another aspect, the invention provides a hybrid neurotoxin comprising a clostridial light chain and a selective ganglioside binding moiety, wherein the selective ganglioside binding moiety is not a clostridial H_{CC} or H_{C} domain, and wherein said selective ganglioside binding moiety binds to GM1 for use in treating sialorrhea (or excessive salivation or drooling).

In another aspect, the invention provides a hybrid neurotoxin comprising a clostridial light chain and a selective ganglioside binding moiety, wherein the selective ganglioside binding moiety is not a clostridial H_{CC} or H_{C} domain, and wherein said selective ganglioside binding moiety binds to one or more gangliosides selected from NeuAc GM3, NeuGc GM3, GM2, GM1, GD3 and GD2, for use in treating cancer.

In another aspect, the present invention provides a method of treatment comprising the administration of a therapeutically effective amount of a hybrid neurotoxin or a pharmaceutical composition according to the invention to a patient in need thereof.

In another aspect, the present invention provides a method of treating a limb disorder associated with unwanted neuronal activity, comprising the administration of a therapeutically effective amount of a hybrid neurotoxin comprising a clostridial light chain and a selective ganglioside binding moiety, wherein the selective ganglioside binding moiety is not a clostridial H_{CC} or H_{C} domain, and wherein said selective ganglioside binding moiety binds to one or more gangliosides selected from GM1a, GM1b, GD1a and GalNAc-GD1a to a patient in need thereof.

In another aspect, the present invention provides a method of treating a head or neck disorder associated with unwanted neuronal activity, comprising the administration of a therapeutically effective amount of a hybrid neurotoxin comprising a clostridial light chain and a selective ganglioside binding moiety, wherein the selective ganglioside binding moiety is not a clostridial H_{CC} or H_{C} domain, and wherein said selective ganglioside binding moiety binds to one or more gangliosides selected from GT1a and GQ1b to a patient in need thereof.

In another aspect, the present invention provides a method of treating sialorrhea (or excessive salivation or drooling), comprising the administration of a therapeutically effective amount of a hybrid neurotoxin comprising a clostridial light chain and a selective ganglioside binding moiety, wherein the selective ganglioside binding moiety is not a clostridial H_{CC} or H_{C} domain, and wherein said selective ganglioside binding moiety binds to GM1 to a patient in need thereof.

In another aspect, the present invention provides a method of treating cancer, comprising the administration of a therapeutically effective amount of a hybrid neurotoxin comprising a clostridial light chain and a selective ganglioside binding moiety, wherein the selective ganglioside binding moiety is not a clostridial H_{CC} or H_{C} domain, and wherein said selective ganglioside binding moiety binds to one or more gangliosides selected from NeuAc GM3, NeuGc GM3, GM2, GM1, GD3 and GD2 to a patient in need thereof.

It shall be understood that all other various features related to the hybrid neurotoxin herein described and preferred embodiments apply mutatis mutandis to the therapeutic and cosmetic aspects of the invention.

It shall also be understood that the pharmaceutical composition according to the invention can be used for the therapeutic and cosmetic purposes of the invention.

The engineered hybrid neurotoxins of the present invention may be formulated for oral, parenteral, continuous infusion, inhalation or topical application. Compositions suitable for injection may be in the form of solutions, suspensions or emulsions, or dry powders which are dissolved or suspended in a suitable vehicle prior to use.

In the case of a hybrid neurotoxin that is to be delivered locally, the hybrid neurotoxin may be formulated as a cream (e.g. for topical application), or for sub-dermal injection.

Local delivery means may include an aerosol, or other spray (e.g. a nebuliser). In this regard, an aerosol formulation of a hybrid neurotoxin enables delivery to the lungs and/or other nasal and/or bronchial or airway passages.

Hybrid neurotoxins of the invention may be administered to a patient by intrathecal or epidural injection in the spinal column at the level of the spinal segment involved in the innervation of an affected organ.

A preferred route of administration is via laparoscopic and/or localised, particularly intramuscular, injection.

The dosage ranges for administration of the neurotoxins of the present invention are those to produce the desired therapeutic effect. It will be appreciated that the dosage range required depends on the precise nature of the hybrid neurotoxin or composition, the route of administration, the nature of the formulation, the age of the patient, the nature, extent or severity of the patient's condition, contraindications, if any, and the judgement of the attending physician. Variations in these dosage levels can be adjusted using standard empirical routines for optimisation.

Fluid dosage forms are typically prepared utilising the hybrid neurotoxin and a pyrogen-free sterile vehicle. The engineered hybrid neurotoxin, depending on the vehicle and concentration used, can be either dissolved or suspended in the vehicle. In preparing solutions the hybrid neurotoxin can be dissolved in the vehicle, the solution being made isotonic if necessary by addition of sodium chloride and sterilised by filtration through a sterile filter using aseptic techniques before filling into suitable sterile vials or ampoules and sealing. Alternatively, if solution stability is adequate, the solution in its sealed containers may be sterilised by autoclaving. Advantageously additives such as buffering, solubilising, stabilising, preservative or bactericidal, suspending or emulsifying agents and or local anaesthetic agents may be dissolved in the vehicle.

Dry powders, which are dissolved or suspended in a suitable vehicle prior to use, may be prepared by filling pre-sterilised ingredients into a sterile container using aseptic technique in a sterile area. Alternatively the ingredients may be dissolved into suitable containers using aseptic technique in a sterile area. The product is then freeze dried and the containers are sealed aseptically.

Parenteral suspensions, suitable for intramuscular, subcutaneous or intradermal injection, are prepared in substantially the same manner, except that the sterile components are suspended in the sterile vehicle, instead of being dissolved and sterilisation cannot be accomplished by filtration. The components may be isolated in a sterile state or alternatively it may be sterilised after isolation, e.g. by gamma irradiation.

Administration in accordance with the present invention may take advantage of a variety of delivery technologies including microparticle encapsulation, viral delivery systems or high-pressure aerosol impingement.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise. Thus, for example, reference to “a clostridial neurotoxin” includes a plurality of such candidate agents and reference to “the clostridial neurotoxin” includes reference to one or more clostridial neurotoxins and equivalents thereof known to those skilled in the art, and so forth.

SEQUENCE INFORMATION
BoNT/A1 - UniProtKB Accession Number P10845 (Clostridium
botulinum)
SEQ ID NO: 1
MPFVNKQFNYKDPVNGVDIAYIKIPNAGQMQPVKAFKIHNKIWVIPERDTFTNPE
EGDLNPPPEAKQVPVSYYDSTYLSTDNEKDNYLKGVTKLFERIYSTDLGRMLLTS
IVRGIPFWGGSTIDTELKVIDTNCINVIQPDGSYRSEELNLVIIGPSADIIQFECKSFG
HEVLNLTRNGYGSTQYIRFSPDFTFGFEESLEVDTNPLLGAGKFATDPAVTLAHE
LIHAGHRLYGIAINPNRVFKVNTNAYYEMSGLEVSFEELRTFGGHDAKFIDSLQE
NEFRLYYYNKFKDIASTLNKAKSIVGTTASLQYMKNVFKEKYLLSEDTSGKFSV
DKLKFDKLYKMLTEIYTEDNFVKFFKVLNRKTYLNFDKAVFKINIVPKVNYTIYD
GFNLRNTNLAANFNGQNTEINNMNFTKLKNFTGLFEFYKLLCVRGIITSKTKSLD
KGYNKALNDLCIKVNNWDLFFSPSEDNFTNDLNKGEEITSDTNIEAAEENISLDLI
QQYYLTFNFDNEPENISIENLSSDIIGQLELMPNIERFPNGKKYELDKYTMFHYLR
AQEFEHGKSRIALTNSVNEALLNPSRVYTFFSSDYVKKVNKATEAAMFLGWVEQ
LVYDFTDETSEVSTTDKIADITIIIPYIGPALNIGNMLYKDDFVGALIFSGAVILLEFI
PEIAIPVLGTFALVSYIANKVLTVQTIDNALSKRNEKWDEVYKYIVTNWLAKVNT
QIDLIRKKMKEALENQAEATKAIINYQYNQYTEEEKNNINFNIDDLSSKLNESINK
AMININKFLNQCSVSYLMNSMIPYGVKRLEDFDASLKDALLKYIYDNRGTLIGQV
DRLKDKVNNTLSTDIPFQLSKYVDNQRLLSTFTEYIKNIINTSILNLRYESNHLIDL
SRYASKINIGSKVNFDPIDKNQIQLFNLESSKIEVILKNAIVYNSMYENFSTSFWIRI
PKYFNSISLNNEYTIINCMENNSGWKVSLNYGEIIWTLQDTQEIKQRVVFKYSQMI
NISDYINRWIFVTITNNRLNNSKIYINGRLIDQKPISNLGNIHASNNIMFKLDGCRD
THRYIWIKYFNLFDKELNEKEIKDLYDNQSNSGILKDFWGDYLQYDKPYYMLNL
YDPNKYVDVNNVGIRGYMYLKGPRGSVMTTNIYLNSSLYRGTKFIIKKYASGNK
DNIVRNNDRVYINVVVKNKEYRLATNASQAGVEKILSALEIPDVGNLSQVVVMK
SKNDQGITNKCKMNLQDNNGNDIGFIGFHQFNNIAKLVASNWYNRQIERSSRTL
GCSWEFIPVDDGWGERPL
BoNT/B1 - UniProtKB Accession Number P10844 (Clostridium
botulinum)
SEQ ID NO: 2
MPVTINNFNYNDPIDNNNIIMMEPPFARGTGRYYKAFKITDRIWIIPERYTFGYKP
EDFNKSSGIFNRDVCEYYDPDYLNTNDKKNIFLQTMIKLFNRIKSKPLGEKLLEMI
INGIPYLGDRRVPLEEFNTNIASVTVNKLISNPGEVERKKGIFANLIIFGPGPVLNEN
ETIDIGIQNHFASREGFGGIMQMKFCPEYVSVFNNVQENKGASIFNRRGYFSDPAL
ILMHELIHVLHGLYGIKVDDLPIVPNEKKFFMQSTDAIQAEELYTFGGQDPSIITPS
TDKSIYDKVLQNFRGIVDRLNKVLVCISDPNININIYKNKFKDKYKFVEDSEGKYS
IDVESFDKLYKSLMFGFTETNIAENYKIKTRASYFSDSLPPVKIKNLLDNEIYTIEE
GFNISDKDMEKEYRGQNKAINKQAYEEISKEHLAVYKIQMCKSVKAPGICIDVD
NEDLFFIADKNSFSDDLSKNERIEYNTQSNYIENDFPINELILDTDLISKIELPSENTE
SLTDFNVDVPVYEKQPAIKKIFTDENTIFQYLYSQTFPLDIRDISLTSSFDDALLFSN
KVYSFFSMDYIKTANKVVEAGLFAGWVKQIVNDFVIEANKSNTMDKIADISLIVP
YIGLALNVGNETAKGNFENAFEIAGASILLEFIPELLIPVVGAFLLESYIDNKNKIIK
TIDNALTKRNEKWSDMYGLIVAQWLSTVNTQFYTIKEGMYKALNYQAQALEEII
KYRYNIYSEKEKSNINIDFNDINSKLNEGINQAIDNINNFINGCSVSYLMKKMIPLA
VEKLLDFDNTLKKNLLNYIDENKLYLIGSAEYEKSKVNKYLKTIMPFDLSIYTND
TILIEMFNKYNSEILNNIILNLRYKDNNLIDLSGYGAKVEVYDGVELNDKNQFKLT
SSANSKIRVTQNQNIIFNSVFLDFSVSFWIRIPKYKNDGIQNYIHNEYTIINCMKNN
SGWKISIRGNRIIWTLIDINGKTKSVFFEYNIREDISEYINRWFFVTITNNLNNAKIY
INGKLESNTDIKDIREVIANGEIIFKLDGDIDRTQFIWMKYFSIFNTELSQSNIEERY
KIQSYSEYLKDFWGNPLMYNKEYYMFNAGNKNSYIKLKKDSPVGEILTRSKYNQ
NSKYINYRDLYIGEKFIIRRKSNSQSINDDIVRKEDYIYLDFFNLNQEWRVYTYKY
FKKEEEKLFLAPISDSDEFYNTIQIKEYDEQPTYSCQLLFKKDEESTDEIGLIGIHRF
YESGIVFEEYKDYFCISKWYLKEVKRKPYNLKLGCNWQFIPKDEGWTE
BoNT/C1 - UniProtKB Accession Number P18640 (Clostridium
botulinum)
SEQ ID NO: 3
MPITINNFNYSDPVDNKNILYLDTHLNTLANEPEKAFRITGNIWVIPDRFSRNSNP
NLNKPPRVTSPKSGYYDPNYLSTDSDKDPFLKEIIKLFKRINSREIGEELIYRLSTDI
PFPGNNNTPINTFDFDVDFNSVDVKTRQGNNWVKTGSINPSVIITGPRENIIDPETS
TFKLTNNTFAAQEGFGALSIISISPRFMLTYSNATNDVGEGRFSKSEFCMDPILILM
HELNHAMHNLYGIAIPNDQTISSVTSNIFYSQYNVKLEYAEIYAFGGPTIDLIPKSA
RKYFEEKALDYYRSIAKRLNSITTANPSSFNKYIGEYKQKLIRKYRFVVESSGEVT
VNRNKFVELYNELTQIFTEFNYAKIYNVQNRKIYLSNVYTPVTANILDDNVYDIQ
NGFNIPKSNLNVLFMGQNLSRNPALRKVNPENMLYLFTKFCHKAIDGRSLYNKT
LDCRELLVKNTDLPFIGDISDVKTDIFLRKDINEETEVIYYPDNVSVDQVILSKNTS
EHGQLDLLYPSIDSESEILPGENQVFYDNRTQNVDYLNSYYYLESQKLSDNVEDF
TFTRSIEEALDNSAKVYTYFPTLANKVNAGVQGGLFLMWANDVVEDFTTNILRK
DTLDKISDVSAIIPYIGPALNISNSVRRGNFTEAFAVTGVTILLEAFPEFTIPALGAF
VIYSKVQERNEIIKTIDNCLEQRIKRWKDSYEWMMGTWLSRIITQFNNISYQMYD
SLNYQAGAIKAKIDLEYKKYSGSDKENIKSQVENLKNSLDVKISEAMNNINKFIR
ECSVTYLFKNMLPKVIDELNEFDRNTKAKLINLIDSHNIILVGEVDKLKAKVNNSF
QNTIPFNIFSYTNNSLLKDIINEYFNNINDSKILSLQNRKNTLVDTSGYNAEVSEEG
DVQLNPIFPFDFKLGSSGEDRGKVIVTQNENIVYNSMYESFSISFWIRINKWVSNL
PGYTIIDSVKNNSGWSIGIISNFLVFTLKQNEDSEQSINFSYDISNNAPGYNKWFFV
TVTNNMMGNMKIYINGKLIDTIKVKELTGINFSKTITFEINKIPDTGLITSDSDNIN
MWIRDFYIFAKELDGKDINILFNSLQYTNVVKDYWGNDLRYNKEYYMVNIDYL
NRYMYANSRQIVFNTRRNNNDFNEGYKIIIKRIRGNTNDTRVRGGDILYFDMTIN
NKAYNLFMKNETMYADNHSTEDIYAIGLREQTKDINDNIIFQIQPMNNTYYYASQ
IFKSNFNGENISGICSIGTYRFRLGGDWYRHNYLVPTVKQGNYASLLESTSTHWG
FVPVSE
BoNT/D - UniProtKB Accession Number P19321 (Clostridium
botulinum)
SEQ ID NO: 4
MTWPVKDFNYSDPVNDNDILYLRIPQNKLITTPVKAFMITQNIWVIPERFSSDTNP
SLSKPPRPTSKYQSYYDPSYLSTDEQKDTFLKGIIKLFKRINERDIGKKLINYLVVG
SPFMGDSSTPEDTFDFTRHTTNIAVEKFENGSWKVTNIITPSVLIFGPLPNILDYTA
SLTLQGQQSNPSFEGFGTLSILKVAPEFLLTFSDVTSNQSSAVLGKSIFCMDPVIAL
MHELTHSLHQLYGINIPSDKRIRPQVSEGFFSQDGPNVQFEELYTFGGLDVEIIPQI
ERSQLREKALGHYKDIAKRLNNINKTIPSSWISNIDKYKKIFSEKYNFDKDNTGNF
VVNIDKFNSLYSDLTNVMSEVVYSSQYNVKNRTHYFSRHYLPVFANILDDNIYTI
RDGFNLTNKGFNIENSGQNIERNPALQKLSSESVVDLFTKVCLRLTKNSRDDSTCI
KVKNNRLPYVADKDSISQEIFENKIITDETNVQNYSDKFSLDESILDGQVPINPEIV
DPLLPNVNMEPLNLPGEEIVFYDDITKYVDYLNSYYYLESQKLSNNVENITLTTS
VEEALGYSNKIYTFLPSLAEKVNKGVQAGLFLNWANEVVEDFTTNIMKKDTLDK
ISDVSVIIPYIGPALNIGNSALRGNFNQAFATAGVAFLLEGFPEFTIPALGVFTFYSS
IQEREKIIKTIENCLEQRVKRWKDSYQWMVSNWLSRITTQFNHINYQMYDSLSYQ
ADAIKAKIDLEYKKYSGSDKENIKSQVENLKNSLDVKISEAMNNINKFIRECSVTY
LFKNMLPKVIDELNKFDLRTKTELINLIDSHNIILVGEVDRLKAKVNESFENTMPF
NIFSYTNNSLLKDIINEYFNSINDSKILSLQNKKNALVDTSGYNAEVRVGDNVQLN
TIYTNDFKLSSSGDKIIVNLNNNILYSAIYENSSVSFWIKISKDLTNSHNEYTIINSIE
QNSGWKLCIRNGNIEWILQDVNRKYKSLIFDYSESLSHTGYTNKWFFVTITNNIM
GYMKLYINGELKQSQKIEDLDEVKLDKTIVFGIDENIDENQMLWIRDFNIFSKELS
NEDINIVYEGQILRNVIKDYWGNPLKFDTEYYIINDNYIDRYIAPESNVLVLVQYP
DRSKLYTGNPITIKSVSDKNPYSRILNGDNIILHMLYNSRKYMIIRDTDTIYATQGG
ECSQNCVYALKLQSNLGNYGIGIFSIKNIVSKNKYCSQIFSSFRENTMLLADIYKP
WRFSFKNAYTPVAVTNYETKLLSTSSFWKFISRDPGWVE
BoNT/E - Accession number WP_003372387 (Clostridium
botulinum)
SEQ ID NO: 5
MPKINSFNYNDPVNDRTILYIKPGGCQEFYKSFNIMKNIWIIPERNVIGTTPQDFHP
PTSLKNGDSSYYDPNYLQSDEEKDRFLKIVTKIFNRINNNLSGGILLEELSKANPY
LGNDNTPDNQFHIGDASAVEIKFSNGSQDILLPNVIIMGAEPDLFETNSSNISLRNN
YMPSNHGFGSIAIVTFSPEYSFRFNDNSMNEFIQDPALTLMHELIHSLHGLYGAKG
ITTKYTITQKQNPLITNIRGTNIEEFLTFGGTDLNIITSAQSNDIYTNLLADYKKIAS
KLSKVQVSNPLLNPYKDVFEAKYGLDKDASGIYSVNINKFNDIFKKLYSFTEFDL
ATKFQVKCRQTYIGQYKYFKLSNLLNDSIYNISEGYNINNLKVNFRGQNANLNPR
IITPITGRGLVKKIIRFCKNIVSVKGIRKSICIEINNGELFFVASENSYNDDNINTPKEI
DDTVTSNNNYENDLDQVILNFNSESAPGLSDEKLNLTIQNDAYIPKYDSNGTSDIE
QHDVNELNVFFYLDAQKVPEGENNVNLTSSIDTALLEQPKIYTFFSSEFINNVNKP
VQAALFVSWIQQVLVDFTTEANQKSTVDKIADISIVVPYIGLALNIGNEAQKGNF
KDALELLGAGILLEFEPELLIPTILVFTIKSFLGSSDNKNKVIKAINNALKERDEKW
KEVYSFIVSNWMTKINTQFNKRKEQMYQALQNQVNAIKTIIESKYNSYTLEEKNE
LTNKYDIKQIENELNQKVSIAMNNIDRFLTESSISYLMKLINEVKINKLREYDENV
KTYLLNYIIQHGSILGESQQELNSMVTDTLNNSIPFKLSSYTDDKILISYFNKFFKRI
KSSSVLNMRYKNDKYVDTSGYDSNININGDVYKYPTNKNQFGIYNDKLSEVNIS
QNDYIIYDNKYKNFSISFWVRIPNYDNKIVNVNNEYTIINCMRDNNSGWKVSLNH
NEIIWTLQDNAGINQKLAFNYGNANGISDYINKWIFVTITNDRLGDSKLYINGNLI
DQKSILNLGNIHVSDNILFKIVNCSYTRYIGIRYFNIFDKELDETEIQTLYSNEPNTN
ILKDFWGNYLLYDKEYYLLNVLKPNNFIDRRKDSTLSINNIRSTILLANRLYSGIK
VKIQRVNNSSTNDNLVRKNDQVYINFVASKTHLFPLYADTATTNKEKTIKISSSG
NRENQVVVMNSVGNNCTMNEKNNNGNNIGLLGFKADTVVASTWYYTHMRDH
TNSNGCFWNFISEEHGWQEK
BoNT/F - UniProtKB Accession Number YP_001390123
(Clostridium botulinum)
SEQ ID NO: 6
MPVVINSFNYNDPVNDDTILYMQIPYEEKSKKYYKAFEIMRNVWIIPERNTIGTDP
SDFDPPASLENGSSAYYDPNYLTTDAEKDRYLKTTIKLFKRINSNPAGEVLLQEIS
YAKPYLGNEHTPINEFHPVTRTTSVNIKSSTNVKSSIILNLLVLGAGPDIFENSSYP
VRKLMDSGGVYDPSNDGFGSINIVTFSPEYEYTFNDISGGYNSSTESFIADPAISLA
HELIHALHGLYGARGVTYKETIKVKQAPLMIAEKPIRLEEFLTFGGQDLNIITSAM
KEKIYNNLLANYEKIATRLSRVNSAPPEYDINEYKDYFQWKYGLDKNADGSYTV
NENKFNEIYKKLYSFTEIDLANKFKVKCRNTYFIKYGFLKVPNLLDDDIYTVSEGF
NIGNLAVNNRGQNIKLNPKIIDSIPDKGLVEKIVKFCKSVIPRKGTKAPPRLCIRVN
NRELFFVASESSYNENDINTPKEIDDTTNLNNNYRNNLDEVILDYNSETIPQISNQT
LNTLVQDDSYVPRYDSNGTSEIEEHNVVDLNVFFYLHAQKVPEGETNISLTSSIDT
ALSEESQVYTFFSSEFINTINKPVHAALFISWINQVIRDFTTEATQKSTFDKIADISL
VVPYVGLALNIGNEVQKENFKEAFELLGAGILLEFVPELLIPTILVFTIKSFIGSSEN
KNKIIKAINNSLMERETKWKEIYSWIVSNWLTRINTQFNKRKEQMYQALQNQVD
AIKTVIEYKYNNYTSDERNRLESEYNINNIREELNKKVSLAMENIERFITESSIFYL
MKLINEAKVSKLREYDEGVKEYLLDYISEHRSILGNSVQELNDLVTSTLNNSIPFE
LSSYTNDKILILYFNKLYKKIKDNSILDMRYENNKFIDISGYGSNISINGDVYIYST
NRNQFGIYSSKPSEVNIAQNNDIIYNGRYQNFSISFWVRIPKYFNKVNLNNEYTIID
CIRNNNSGWKISLNYNKIIWTLQDTAGNNQKLVFNYTQMISISDYINKWIFVTITN
NRLGNSRIYINGNLIDEKSISNLGDIHVSDNILFKIVGCNDTRYVGIRYFKVFDTEL
GKTEIETLYSDEPDPSILKDFWGNYLLYNKRYYLLNLLRTDKSITQNSNFLNINQQ
RGVYQKPNIFSNTRLYTGVEVIIRKNGSTDISNTDNFVRKNDLAYINVVDRDVEY
RLYADISIAKPEKIIKLIRTSNSNNSLGQIIVMDSIGNNCTMNFQNNNGGNIGLLGF
HSNNLVASSWYYNNIRKNTSSNGCFWSFISKEHGWQEN
BoNT/G - UniProtKB Accession Number WP_039635782
(Clostridium botulinum)
SEQ ID NO: 7
MPVNIKNFNYNDPINNDDIIMMEPFNDPGPGTYYKAFRIIDRIWIVPERFTYGFQP
DQFNASTGVFSKDVYEYYDPTYLKTDAEKDKFLKTMIKLFNRINSKPSGQRLLD
MIVDAIPYLGNASTPPDKFAANVANVSINKKIIQPGAEDQIKGLMTNLIIFGPGPVL
SDNFTDSMIMNGHSPISEGFGARMMIRFCPSCLNVFNNVQENKDTSIFSRRAYFA
DPALTLMHELIHVLHGLYGIKISNLPITPNTKEFFMQHSDPVQAEELYTFGGHDPS
VISPSTDMNIYNKALQNFQDIANRLNIVSSAQGSGIDISLYKQIYKNKYDFVEDPN
GKYSVDKDKFDKLYKALMFGFTETNLAGEYGIKTRYSYFSEYLPPIKTEKLLDNT
IYTQNEGFNIASKNLKTEFNGQNKAVNKEAYEEISLEHLVIYRIAMCKPVMYKNT
GKSEQCIIVNNEDLFFIANKDSFSKDLAKAETIAYNTQNNTIENNFSIDQLILDNDL
SSGIDLPNENTEPFTNFDDIDIPVYIKQSALKKIFVDGDSLFEYLHAQTFPSNIENLQ
LTNSLNDALRNNNKVYTFFSTNLVEKANTVVGASLFVNWVKGVIDDFTSESTQK
STIDKVSDVSIIIPYIGPALNVGNETAKENFKNAFEIGGAAILMEFIPELIVPIVGFFT
LESYVGNKGHIIMTISNALKKRDQKWTDMYGLIVSQWLSTVNTQFYTIKERMYN
ALNNQSQAIEKIIEDQYNRYSEEDKMNINIDFNDIDFKLNQSINLAINNIDDFINQC
SISYLMNRMIPLAVKKLKDFDDNLKRDLLEYIDTNELYLLDEVNILKSKVNRHLK
DSIPFDLSLYTKDTILIQVFNNYISNISSNAILSLSYRGGRLIDSSGYGATMNVGSD
VIFNDIGNGQFKLNNSENSNITAHQSKFVVYDSMFDNFSINFWVRTPKYNNNDIQ
TYLQNEYTIISCIKNDSGWKVSIKGNRIIWTLIDVNAKSKSIFFEYSIKDNISDYINK
WFSITITNDRLGNANIYINGSLKKSEKILNLDRINSSNDIDFKLINCTDTTKFVWIK
DFNIFGRELNATEVSSLYWIQSSTNTLKDFWGNPLRYDTQYYLFNQGMQNIYIKY
FSKASMGETAPRTNFNNAAINYQNLYLGLRFIIKKASNSRNINNDNIVREGDYIYL
NIDNISDESYRVYVLVNSKEIQTQLFLAPINDDPTFYDVLQIKKYYEKTTYNCQIL
CEKDTKTFGLFGIGKFVKDYGYVWDTYDNYFCISQWYLRRISENINKLRLGCNW
QFIPVDEGWTE
TeNT - UniProtKB Accession Number P04958 (Clostridium
tetani)
SEQ ID NO: 8
MPITINNFRYSDPVNNDTIIMMEPPYCKGLDIYYKAFKITDRIWIVPERYEFGTKPE
DFNPPSSLIEGASEYYDPNYLRTDSDKDRFLQTMVKLFNRIKNNVAGEALLDKIIN
AIPYLGNSYSLLDKFDTNSNSVSFNLLEQDPSGATTKSAMLTNLIIFGPGPVLNKN
EVRGIVLRVDNKNYFPCRDGFGSIMQMAFCPEYVPTFDNVIENITSLTIGKSKYFQ
DPALLLMHELIHVLHGLYGMQVSSHEIIPSKQEIYMQHTYPISAEELFTFGGQDAN
LISIDIKNDLYEKTLNDYKAIANKLSQVTSCNDPNIDIDSYKQIYQQKYQFDKDSN
GQYIVNEDKFQILYNSIMYGFTEIELGKKFNIKTRLSYFSMNHDPVKIPNLLDDTIY
NDTEGFNIESKDLKSEYKGQNMRVNTNAFRNVDGSGLVSKLIGLCKKIIPPTNIRE
NLYNRTASLTDLGGELCIKIKNEDLTFIAEKNSFSEEPFQDEIVSYNTKNKPLNFN
YSLDKIIVDYNLQSKITLPNDRTTPVTKGIPYAPEYKSNAASTIEIHNIDDNTIYQY
LYAQKSPTTLQRITMTNSVDDALINSTKIYSYFPSVISKVNQGAQGILFLQWVRDII
DDFTNESSQKTTIDKISDVSTIVPYIGPALNIVKQGYEGNFIGALETTGVVLLLEYIP
EITLPVIAALSIAESSTQKEKIIKTIDNFLEKRYEKWIEVYKLVKAKWLGTVNTQF
QKRSYQMYRSLEYQVDAIKKIIDYEYKIYSGPDKEQIADEINNLKNKLEEKANKA
MININIFMRESSRSFLVNQMINEAKKQLLEFDTQSKNILMQYIKANSKFIGITELKK
LESKINKVFSTPIPFSYSKNLDCWVDNEEDIDVILKKSTILNLDINNDIISDISGFNSS
VITYPDAQLVPGINGKAIHLVNNESSEVIVHKAMDIEYNDMFNNFTVSFWLRVPK
VSASHLEQYGTNEYSIISSMKKHSLSIGSGWSVSLKGNNLIWTLKDSAGEVRQITF
RDLPDKFNAYLANKWVFITITNDRLSSANLYINGVLMGSAEITGLGAIREDNNITL
KLDRCNNNNQYVSIDKFRIFCKALNPKEIEKLYTSYLSITFLRDFWGNPLRYDTEY
YLIPVASSSKDVQLKNITDYMYLTNAPSYTNGKLNIYYRRLYNGLKFIIKRYTPN
NEIDSFVKSGDFIKLYVSYNNNEHIVGYPKDGNAFNNLDRILRVGYNAPGIPLYK
KMEAVKLRDLKTYSVQLKLYDDKNASLGLVGTHNGQIGNDPNRDILIASNWYF
NHLKDKILGCDWYFVPTDEGWTND
Cholera toxin B subunit (Vibrio cholera)
SEQ ID NO: 9
MIKLKFGVFFTVLLSSAYAHGTPQNITDLCAEYHNTQIYTLNDKIFSYTESLAGKR
EMAIITFKNGAIFQVEVPGSQHIDSQKKAIERMKDTLRIAYLTEAKVEKLCVWNN
KTPHAIAAISMAN
Cholera toxin A subunit (Vibrio cholera)
SEQ ID NO: 10
MVKIIFVFFIFLSSFSYANDDKLYRADSRPPDEIKQSGGLMPRGQSEYFDRGTQMN
INLYDHARGTQTGFVRHDDGYVSTSISLRSAHLVGQTILSGHSTYYIYVIATAPN
MFNVNDVLGAYSPHPDEQEVSALGGIPYSQIYGWYRVHFGVLDEQLHRNRGYR
DRYYSNLDIAPAADGYGLAGFPPEHRAWREEPWIHHAPPGCGNAPRSSMSNTCD
EKTQSLGVKFLDEYQSKVKRQIFSGYQSDIDTHNRIKDEL
BoNT/A1(0)-CtxBCP (Artificial sequence)
SEQ ID NO: 11
MGSMEFVNKQFNYKDPVNGVDIAYIKIPNAGQMQPVKAFKIHNKIWVIPERDTF
TNPEEGDLNPPPEAKQVPVSYYDSTYLSTDNEKDNYLKGVTKLFERIYSTDLGR
MLLTSIVRGIPFWGGSTIDTELKVIDTNCINVIQPDGSYRSEELNLVIIGPSADIIQFE
CKSFGHEVLNLTRNGYGSTQYIRFSPDFTFGFEESLEVDTNPLLGAGKFATDPAV
TLAHQLIYAGHRLYGIAINPNRVFKVNTNAYYEMSGLEVSFEELRTFGGHDAKFI
DSLQENEFRLYYYNKFKDIASTLNKAKSIVGTTASLQYMKNVFKEKYLLSEDTSG
KFSVDKLKFDKLYKMLTEIYTEDNFVKFFKVLNRKTYLNFDKAVFKINIVPKVN
YTIYDGFNLRNTNLAANFNGQNTEINNMNFTKLKNFTGLFEFYKLLCVDGIITSK
TKSDDDDKTPQNITDLCAEYHNTQIHTLNDKIFSYTESLAGKREMAIITFKNGATF
QVEVPGSQHIDSQKKAIERMKDTLRIAYLTEAKVEKLCVWNNKTPHAIAAISMA
NSGGGGSGGGGSGGGGSPRGSALNLQCIKVNNWDLFFSPSEDNFTNDLNKGEEI
TSDTNIEAAEENISLDLIQQYYLTFNFDNEPENISIENLSSDIIGQLELMPNIERFPNG
KKYELDKYTMFHYLRAQEFEHGKSRIALTNSVNEALLNPSRVYTFFSSDYVKKV
NKATEAAMFLGWVEQLVYDFTDETSEVSTTDKIADITIIIPYIGPALNIGNMLYKD
DFVGALIFSGAVILLEFIPEIAIPVLGTFALVSYIANKVLTVQTIDNALSKRNEKWD
EVYKYIVTNWLAKVNTQIDLIRKKMKEALENQAEATKAIINYQYNQYTEEEKNN
INFNIDDLSSKLNESINKAMININKFLNQCSVSYLMNSMIPYGVKRLEDFDASLKD
ALLKYIYDNRGTLIGQVDRLKDKVNNTLSTDIPFQLSKYVDNQRLLSTFTEYIKNI
INTSILNLRYESNHLIDLSRYASKINIGSKVNFDPIDKNQIQLFNLESSKIEVILKNAI
VYNSMYENFSTSFWIRIPKYFNSISLNNEYTIINCMENNSGWKVSLNYGEIIWTLQ
DTQEIKQRVVFKYSQMINISDYINRWIFVTITNNRLNNSKIYINGRLIDQKPISNLG
NIHASNNIMFKLDGCRDTHRYIWIKYFNLFDKELNEKEIKDLYDNQSNSGILKDF
WGDYLQYDKPYYMLNLYDPNKYVDVNNVGIRGYMYLKGPRGSVMTTNIYLNS
SLYRGTKFIIKKYASGNKDNIVRNNDRVYINVVVKNKEYRLATNASQAGVEKILS
ALEIPDVGNLSQVVVMKSKNDQGITNKCKMNLQDNNGNDIGFIGFHQFNNIAKL
VASNWYNRQIERSSRTLGCSWEFIPVDDGWGERPLRKSFHHHHHH

The invention will now be described, by way of example only, with reference to the following Figures and Examples.

### FIGURES

FIG. 1: Exemplars of Ctx-BoNT hybrid neurotoxins

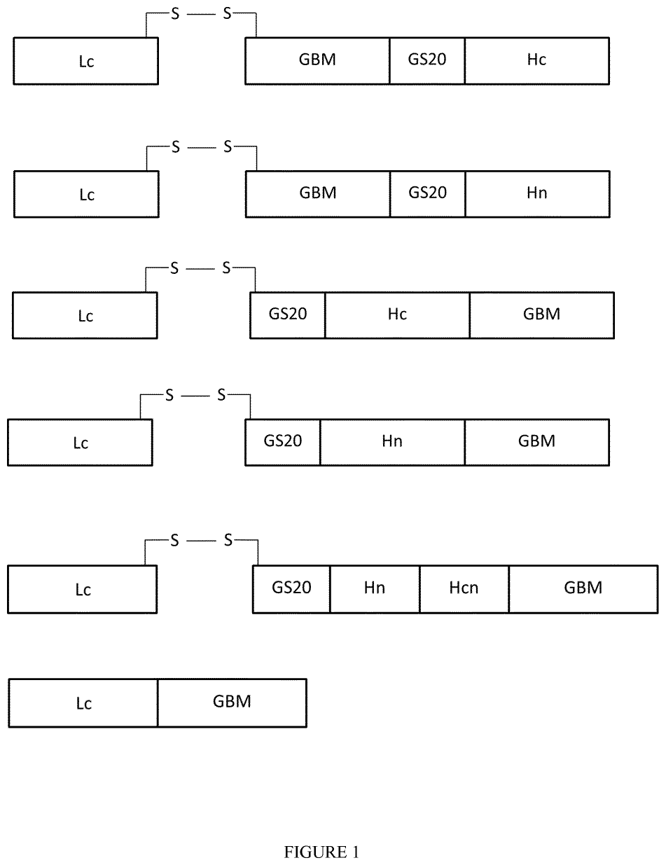

FIG. 2: Fractions analysed by SDS-PAGE of the HisTrap HP capture column. Target construct elutes in fractions E3-F6 (250 mM-500 mM Imidazole).

FIG. 3: Fractions analysed by SDS-PAGE of second chromatography step, anion exchange. Target protein eluted in fractions 13-30 (across an increasing NaCl concentration).

FIG. 4: Fractions analysed by SDS-PAGE after activation with enterokinase. The analysis shows the protein is not stable prior to proteolytic activation, though some of the construct does appear to remain intact. Enterokinase activation does cleave the construct between the light and heavy chain and from the SDS-PAGE analysis suggests at least some of the product is of the predicted composition, i.e intact light and heavy chain as well as attached GS20 and CtxB in the central presentation.

FIG. 5: western blot analysis after activation with enterokinase. 5A—Blots treated with monoclonal tetra his antibody, secondary anti-mouse conjugate; 5B—Blots treated with anti-LcA antidody and secondary anti-rabbit conjugate.

FIG. 6: assessment of free Ctx-B, BoNT/A1(0)-CtxBCP and BoNT/A1(0) in a GM1 competitive binding assay

### EXAMPLES

### Example 1—Expression and Purification of BoNT/A1(0)-CtxBCP

A codon optimised (for E. coli) construct was designed based on CtxB primary protein sequence (residues 22 to 103 of SEQ ID NO: 11), and sub-cloned into endonegative BoNT/A into a pJ401 plasmid with a T5 promotor to produce a centrally presented construct (BoNT/A1(0)-CtxBCP), with an enterokinase activation site (EK), a GS20 linker and a C-terminal His-tag: L_{c}A(0)-EK-CtxB-GS20-HSA-6HT.

The construct was transformed into E. coli strain BL21 (DE3) in mTB medium (Tryptone 12 g/l, Yeast Extract 24 g/l, Dipotassium phosphate 9.4 g/l, Monopotassium phosphate 2.2 g/l, Melford) supplemented with Glycerol (0.4%, Sigma), Glucosamine (0.2%, Sigma) and 30 μg/ml Kan (Sigma). An individual colony was picked to inoculate a vial of microbank beads. The inoculated beads were stored at −80° C. until required. One bead was used to inoculate 100 ml of the mTB media at 37° C. When the absorbance at 600 nm reached 4.6, 10 ml of the 100 ml culture was added to 1 L of media in a 2 L flask and the culture was grown at 37° C. to OD600≥1.0. The temperature was reduced to 16° C. and the culture was allowed to cool for 1 hour before adding IPTG to a final concentration of 1 mM. Induction continued for 20 hours. The culture was then harvested by centrifugation at 6000 g for 20 minutes at 4° C. Spent media was decanted and the pellet was frozen and stored at −80° C. until required.

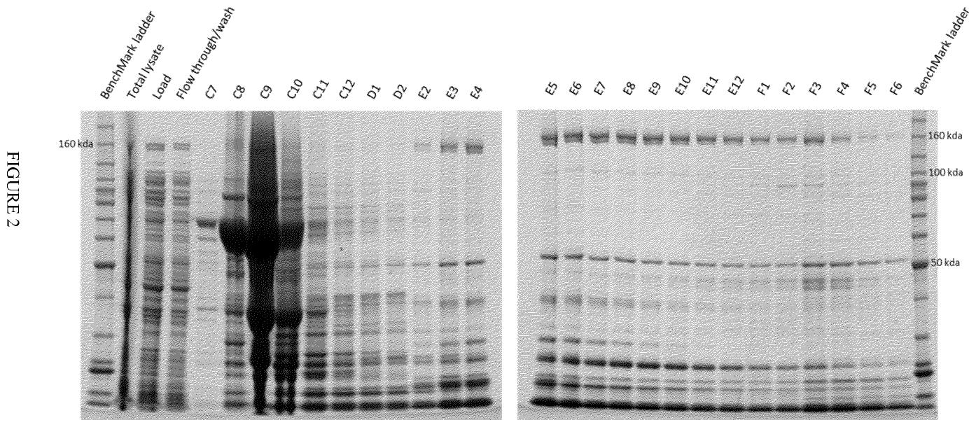

The cell pellet was thawed and resuspended in 6 ml/g lysis buffer (50 mM Tris pH 8.0, 200 mM NaCl). The cells were lysed by homogenisor by a single pass at 20 kpsi. Cell debris and insoluble material was cleared by centrifugation at 30,000 g for 30 minutes. The supernatant was collected and loaded onto a 5 ml HisTrap column (pre-charged with Ni²⁺ and equilibrated with lysis buffer. After loading the column was washed for 50 ml with lysis buffer before eluting the protein across a step-wise gradient of increasing imidazole concentration of 25 ml 40 mM, 50 ml 80 mM, 25 ml 125 mM, 25 ml 250 mM and 25 ml 500 mM. 2.5 ml fractions were collected throughout, and the location of the target chimera was determined by SDS-PAGE (FIG. 2).

Fractions E3-F6 (250 mM-500 mM Imidazole) containing the target protein were pooled and desalted using a 53 ml 26/10 desalt column. The material was desalted into QHP binding buffer (50 mM Tris pH 8.0). The buffer exchanged material was kept as one pool and further processed by anion exchange.

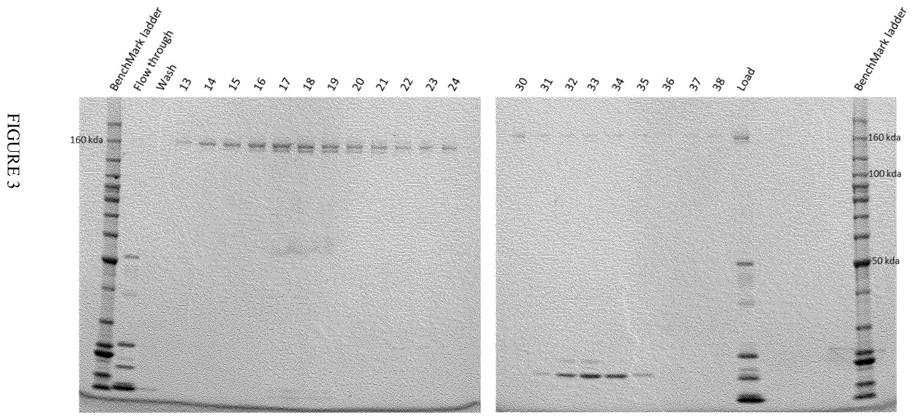

A 5 ml HiTrap QHP column was used to further purify the chimera. The column was pre-equilibrated in binding buffer (50 mM Tris pH 8.0) before loading the desalt pool. The column was washed for 25 ml with binding buffer before eluting the protein over a linear gradient from 0 to 350 mM NaCl over 100 ml. The column was then washed with a high salt step of 350 mM-1 M NaCl over 25 ml. 2.5 ml fractions were collected throughout and analysed by SDS-PAGE to determine which fractions contained the target protein (FIG. 3).

Fractions 13-30 containing the target protein were pooled and concentrated before activation with enterokinase for 18 hours at 4° C. and the reaction was terminated with the addition of AEBSF.

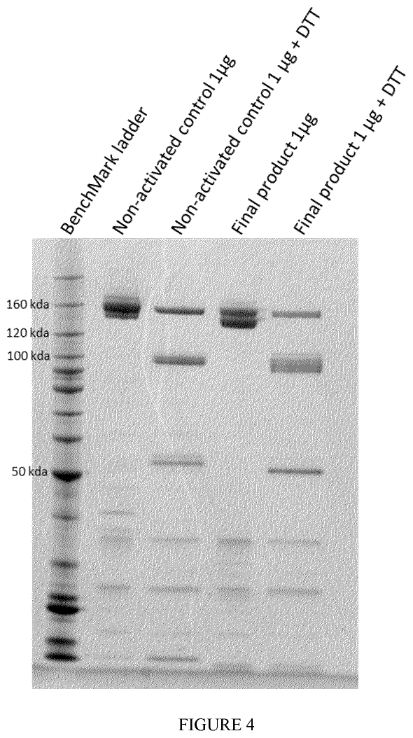

This final material was analysed by SDS-PAGE (FIG. 4).

The analysis shows the protein is not stable prior to proteolytic activation, though some of the construct does appear to remain intact. Enterokinase activation does cleave the construct between the light and heavy chain and from the SDS-PAGE analysis suggests at least some of the product is of the predicted composition, i.e intact light and heavy chain as well as attached GS20 and CtxB in the central presentation.

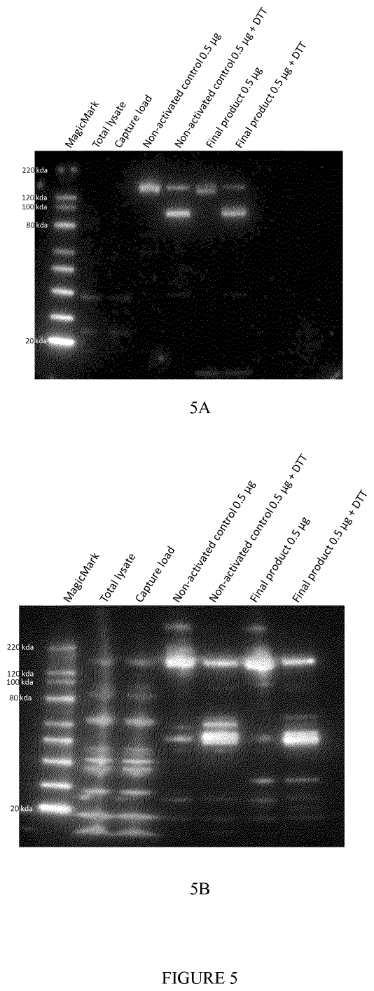

Samples were also analysed by western blot in order to confirm the presence of the light chain and the his-tag (FIG. 5). Protein was transferred from gel to nitrocellulose membrane using a Bio-Rad trans-blot turbo transfer system. The blots were blocked in PBST 0.5% BSA. Blots were either treated with monoclonal tetra his antibody, secondary anti-mouse conjugate or they were treated with anti-LcA and secondary anti-rabbit. Super signal substrate was used to generate signal and detected in a Pxi 4. The western blot shows a positive signal for full length target as well as product related truncates.

### Example 2—Binding of BoNT/A1(0)-CtxBCP to GM1

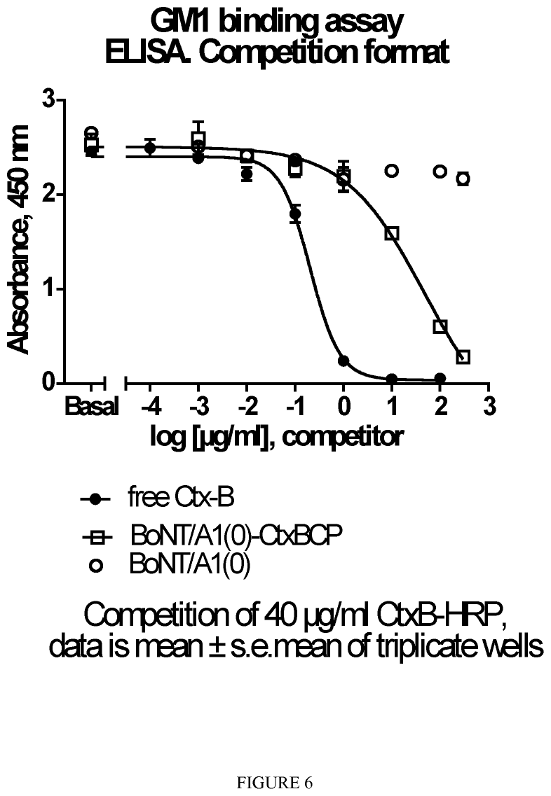

Briefly, a clear F96 Maxisorp plate was coated with 100 ng/ml GM1 overnight, blocked with 2% BSA-PBS solution and preincubated with free cholera toxin B subunit (free Ctx-B), BoNT/A1(0)-CtxBCP or BoNT/A1(0) at the indicated concentrations. Plates were further incubated with 40 μg/ml cholera toxin B subunit conjugated to horseradish peroxidase (Ctx-B-HRP). Activity of the HRP on the plate following washing was determined with a developing solution, and absorbance at 450 nm determined following the stop of the reaction. Data is mean±s.e.mean of triplicate wells (FIG. 6).

FIG. 6 shows that:
BoNT/A1(0) did not compete Ctx-B (Ctx-B-HRP) to GM1 binding, as expected;
Free Ctx-B did compete Ctx-B-HRP, as expected, and with a pEC₅₀ of 0.2 μg/ml;
BoNT/A1(0)-CtxBCP was able to compete Ctx-B-HRP exhibiting a pEC₅₀ about 100-fold lower than free Ctx-B (49 μg/ml).

In conclusion, the addition of a Ctx-B domain confers BoNT/A1(0) with the ability to bind to GM1, a ganglioside which is not a natural receptor for BoNT/A1(0).

1. A hybrid neurotoxin comprising a clostridial light chain (L) and a selective ganglioside binding moiety, wherein said selective ganglioside binding moiety is not a clostridial H_{CC} or a H_{C} domain.

2. A hybrid neurotoxin according to claim 1, wherein said hybrid neurotoxin further comprises a translocation moeity.

3. A hybrid neurotoxin according to claim 2, wherein said translocation moiety is selected from the group consisting of a clostridial H_{N} domain, a Cholera toxin A2 subunit (CtxA2), and cell penetrating peptides.

4. A hybrid neurotoxin according to claim 2 or 3, wherein said translocation moiety is a clostridial H_{N} domain, and wherein said hybrid neurotoxin comprises an activation site between the light chain and the clostridial H_{N} domain.

5. A hybrid neurotoxin according to any one of claims 1 to 4, wherein said hybrid neurotoxin further comprises a clostridial H_{CN} and/or a H_{CC} domain.

6. A hybrid neurotoxin according to any one of claims 1 to 5, wherein said selective ganglioside binding moiety binds to one or more gangliosides selected from the group consisting of GM1 such as GM1a or GM1b, GM2, GM3 such as NeuAc GM3 or NeuGc GM3, GM4, GD1a, GalNAc-GD1a, GT1a, GT1b, GQ1b, GD2, GD3, and any combination thereof.

7. A hybrid neurotoxin according to claim 6, wherein said selective ganglioside binding moiety binds to GM1, and wherein said selective ganglioside binding moiety comprises one or more Cholera toxin B subunits (CtxB) or E. coli heat labile enterotoxin (LT).

8. A hybrid neurotoxin according to claim 7, wherein said selective ganglioside binding moiety comprises one or more Cholera toxin B subunits (CtxB) and said light chain is covalently bound to said one or more Cholera toxin B subunits (CtxB).

9. A hybrid neurotoxin according to claim 7, wherein said selective ganglioside binding moiety comprises one or more Cholera toxin B subunits (CtxB) and said hybrid neurotoxin comprises a Cholera toxin A2 subunit (CtxA2), wherein said CtxA2 is covalently bound to said clostridial light chain, and wherein said CtxB forms a non covalent link with said clostridial light chain.

10. A hybrid neurotoxin according to any one of claims 1 to 9, wherein said clostridial light chain is from a BoNT type A, type B, type Cl, type D, type E, type F or type G or a TeNT.

11. A nucleotide sequence encoding a hybrid neurotoxin according to any one of claims 1 to 10.

12. A vector comprising a nucleotide sequence according to claim 11.

13. A cell comprising a nucleotide sequence according to claim 11 or a vector according to claim 12.

14. A pharmaceutical composition comprising a hybrid neurotoxin according to any one of claims 1 to 10.

15. A hybrid neurotoxin according to any one of claims 1 to 10 or a pharmaceutical composition according to claim 14 for use in therapy.

16. A hybrid neurotoxin according to any one of claims 1 to 10, or a pharmaceutical composition according to claim 14, for use in treating a limb disorder associated with unwanted neuronal activity, wherein said selective ganglioside binding moiety binds to one or more gangliosides selected from GM1a, GM1b, GD1a and GalNAc-GD1a.

17. A hybrid neurotoxin according to any one of claims 1 to 10, or a pharmaceutical composition according to claim 14, for use in treating a head or neck disorder associated with unwanted neuronal activity, wherein said selective ganglioside binding moiety binds to one or more gangliosides selected from GT1a and GQ1b.

18. A hybrid neurotoxin according to any one of claims 1 to 10, or a pharmaceutical composition according to claim 14, for use in treating sialorrhea, wherein said selective ganglioside binding moiety binds to the ganglioside moiety GM1.

19. A hybrid neurotoxin according to any one of claims 1 to 10, or a pharmaceutical composition according to claim 14, for use in treating cancer, wherein said selective ganglioside binding moiety binds to one or more gangliosides selected from NeuAc GM3, NeuGc GM3, GM2, GM1, GD3 and GD2.

20. Non-therapeutic use of a hybrid neurotoxin according to any one of claims 1 to 10 or of a pharmaceutical composition according to claim 14, for treating an aesthetic condition.


SEQUENCE LISTING (11 sequences)

SEQ ID NO: 1 - protein, 1296 aa, Clostridium botulinum
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 2 - protein, 1291 aa, Clostridium botulinum
MPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCKSVKAPGICIDVDNEDLFFIADKNSFSDDLSKNERIEYNTQSNYIENDFPINELILDTDLISKIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIVNDFVIEANKSNTMDKIADISLIVPYIGLALNVGNETAKGNFENAFEIAGASILLEFIPELLIPVVGAFLLESYIDNKNKIIKTIDNALTKRNEKWSDMYGLIVAQWLSTVNTQFYTIKEGMYKALNYQAQALEEIIKYRYNIYSEKEKSNINIDFNDINSKLNEGINQAIDNINNFINGCSVSYLMKKMIPLAVEKLLDFDNTLKKNLLNYIDENKLYLIGSAEYEKSKVNKYLKTIMPFDLSIYTNDTILIEMFNKYNSEILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEEKLFLAPISDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 3 - protein, 1291 aa, Clostridium botulinum
MPITINNFNYSDPVDNKNILYLDTHLNTLANEPEKAFRITGNIWVIPDRFSRNSNPNLNKPPRVTSPKSGYYDPNYLSTDSDKDPFLKEIIKLFKRINSREIGEELIYRLSTDIPFPGNNNTPINTFDFDVDFNSVDVKTRQGNNWVKTGSINPSVIITGPRENIIDPETSTFKLTNNTFAAQEGFGALSIISISPRFMLTYSNATNDVGEGRFSKSEFCMDPILILMHELNHAMHNLYGIAIPNDQTISSVTSNIFYSQYNVKLEYAEIYAFGGPTIDLIPKSARKYFEEKALDYYRSIAKRLNSITTANPSSFNKYIGEYKQKLIRKYRFVVESSGEVTVNRNKFVELYNELTQIFTEFNYAKIYNVQNRKIYLSNVYTPVTANILDDNVYDIQNGFNIPKSNLNVLFMGQNLSRNPALRKVNPENMLYLFTKFCHKAIDGRSLYNKTLDCRELLVKNTDLPFIGDISDVKTDIFLRKDINEETEVIYYPDNVSVDQVILSKNTSEHGQLDLLYPSIDSESEILPGENQVFYDNRTQNVDYLNSYYYLESQKLSDNVEDFTFTRSIEEALDNSAKVYTYFPTLANKVNAGVQGGLFLMWANDVVEDFTTNILRKDTLDKISDVSAIIPYIGPALNISNSVRRGNFTEAFAVTGVTILLEAFPEFTIPALGAFVIYSKVQERNEIIKTIDNCLEQRIKRWKDSYEWMMGTWLSRIITQFNNISYQMYDSLNYQAGAIKAKIDLEYKKYSGSDKENIKSQVENLKNSLDVKISEAMNNINKFIRECSVTYLFKNMLPKVIDELNEFDRNTKAKLINLIDSHNIILVGEVDKLKAKVNNSFQNTIPFNIFSYTNNSLLKDIINEYFNNINDSKILSLQNRKNTLVDTSGYNAEVSEEGDVQLNPIFPFDFKLGSSGEDRGKVIVTQNENIVYNSMYESFSISFWIRINKWVSNLPGYTIIDSVKNNSGWSIGIISNFLVFTLKQNEDSEQSINFSYDISNNAPGYNKWFFVTVTNNMMGNMKIYINGKLIDTIKVKELTGINFSKTITFEINKIPDTGLITSDSDNINMWIRDFYIFAKELDGKDINILFNSLQYTNVVKDYWGNDLRYNKEYYMVNIDYLNRYMYANSRQIVFNTRRNNNDFNEGYKIIIKRIRGNTNDTRVRGGDILYFDMTINNKAYNLFMKNETMYADNHSTEDIYAIGLREQTKDINDNIIFQIQPMNNTYYYASQIFKSNFNGENISGICSIGTYRFRLGGDWYRHNYLVPTVKQGNYASLLESTSTHWGFVPVSE

SEQ ID NO: 4 - protein, 1276 aa, Clostridium botulinum
MTWPVKDFNYSDPVNDNDILYLRIPQNKLITTPVKAFMITQNIWVIPERFSSDTNPSLSKPPRPTSKYQSYYDPSYLSTDEQKDTFLKGIIKLFKRINERDIGKKLINYLVVGSPFMGDSSTPEDTFDFTRHTTNIAVEKFENGSWKVTNIITPSVLIFGPLPNILDYTASLTLQGQQSNPSFEGFGTLSILKVAPEFLLTFSDVTSNQSSAVLGKSIFCMDPVIALMHELTHSLHQLYGINIPSDKRIRPQVSEGFFSQDGPNVQFEELYTFGGLDVEIIPQIERSQLREKALGHYKDIAKRLNNINKTIPSSWISNIDKYKKIFSEKYNFDKDNTGNFVVNIDKFNSLYSDLTNVMSEVVYSSQYNVKNRTHYFSRHYLPVFANILDDNIYTIRDGFNLTNKGFNIENSGQNIERNPALQKLSSESVVDLFTKVCLRLTKNSRDDSTCIKVKNNRLPYVADKDSISQEIFENKIITDETNVQNYSDKFSLDESILDGQVPINPEIVDPLLPNVNMEPLNLPGEEIVFYDDITKYVDYLNSYYYLESQKLSNNVENITLTTSVEEALGYSNKIYTFLPSLAEKVNKGVQAGLFLNWANEVVEDFTTNIMKKDTLDKISDVSVIIPYIGPALNIGNSALRGNFNQAFATAGVAFLLEGFPEFTIPALGVFTFYSSIQEREKIIKTIENCLEQRVKRWKDSYQWMVSNWLSRITTQFNHINYQMYDSLSYQADAIKAKIDLEYKKYSGSDKENIKSQVENLKNSLDVKISEAMNNINKFIRECSVTYLFKNMLPKVIDELNKFDLRTKTELINLIDSHNIILVGEVDRLKAKVNESFENTMPFNIFSYTNNSLLKDIINEYFNSINDSKILSLQNKKNALVDTSGYNAEVRVGDNVQLNTIYTNDFKLSSSGDKIIVNLNNNILYSAIYENSSVSFWIKISKDLTNSHNEYTIINSIEQNSGWKLCIRNGNIEWILQDVNRKYKSLIFDYSESLSHTGYTNKWFFVTITNNIMGYMKLYINGELKQSQKIEDLDEVKLDKTIVFGIDENIDENQMLWIRDFNIFSKELSNEDINIVYEGQILRNVIKDYWGNPLKFDTEYYIINDNYIDRYIAPESNVLVLVQYPDRSKLYTGNPITIKSVSDKNPYSRILNGDNIILHMLYNSRKYMIIRDTDTIYATQGGECSQNCVYALKLQSNLGNYGIGIFSIKNIVSKNKYCSQIFSSFRENTMLLADIYKPWRFSFKNAYTPVAVTNYETKLLSTSSFWKFISRDPGWVE

SEQ ID NO: 5 - protein, 1252 aa, Clostridium botulinum
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHGFGSIAIVTFSPEYSFRFNDNSMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKLINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTLQDNAGINQKLAFNYGNANGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNNFIDRRKDSTLSINNIRSTILLANRLYSGIKVKIQRVNNSSTNDNLVRKNDQVYINFVASKTHLFPLYADTATTNKEKTIKISSSGNRFNQVVVMNSVGNNCTMNFKNNNGNNIGLLGFKADTVVASTWYYTHMRDHTNSNGCFWNFISEEHGWQEK

SEQ ID NO: 6 - protein, 1278 aa, Clostridium botulinum
MPVVINSFNYNDPVNDDTILYMQIPYEEKSKKYYKAFEIMRNVWIIPERNTIGTDPSDFDPPASLENGSSAYYDPNYLTTDAEKDRYLKTTIKLFKRINSNPAGEVLLQEISYAKPYLGNEHTPINEFHPVTRTTSVNIKSSTNVKSSIILNLLVLGAGPDIFENSSYPVRKLMDSGGVYDPSNDGFGSINIVTFSPEYEYTFNDISGGYNSSTESFIADPAISLAHELIHALHGLYGARGVTYKETIKVKQAPLMIAEKPIRLEEFLTFGGQDLNIITSAMKEKIYNNLLANYEKIATRLSRVNSAPPEYDINEYKDYFQWKYGLDKNADGSYTVNENKFNEIYKKLYSFTEIDLANKFKVKCRNTYFIKYGFLKVPNLLDDDIYTVSEGFNIGNLAVNNRGQNIKLNPKIIDSIPDKGLVEKIVKFCKSVIPRKGTKAPPRLCIRVNNRELFFVASESSYNENDINTPKEIDDTTNLNNNYRNNLDEVILDYNSETIPQISNQTLNTLVQDDSYVPRYDSNGTSEIEEHNVVDLNVFFYLHAQKVPEGETNISLTSSIDTALSEESQVYTFFSSEFINTINKPVHAALFISWINQVIRDFTTEATQKSTFDKIADISLVVPYVGLALNIGNEVQKENFKEAFELLGAGILLEFVPELLIPTILVFTIKSFIGSSENKNKIIKAINNSLMERETKWKEIYSWIVSNWLTRINTQFNKRKEQMYQALQNQVDAIKTVIEYKYNNYTSDERNRLESEYNINNIREELNKKVSLAMENIERFITESSIFYLMKLINEAKVSKLREYDEGVKEYLLDYISEHRSILGNSVQELNDLVTSTLNNSIPFELSSYTNDKILILYFNKLYKKIKDNSILDMRYENNKFIDISGYGSNISINGDVYIYSTNRNQFGIYSSKPSEVNIAQNNDIIYNGRYQNFSISFWVRIPKYFNKVNLNNEYTIIDCIRNNNSGWKISLNYNKIIWTLQDTAGNNQKLVFNYTQMISISDYINKWIFVTITNNRLGNSRIYINGNLIDEKSISNLGDIHVSDNILFKIVGCNDTRYVGIRYFKVFDTELGKTEIETLYSDEPDPSILKDFWGNYLLYNKRYYLLNLLRTDKSITQNSNFLNINQQRGVYQKPNIFSNTRLYTGVEVIIRKNGSTDISNTDNFVRKNDLAYINVVDRDVEYRLYADISIAKPEKIIKLIRTSNSNNSLGQIIVMDSIGNNCTMNFQNNNGGNIGLLGFHSNNLVASSWYYNNIRKNTSSNGCFWSFISKEHGWQEN

SEQ ID NO: 7 - protein, 1297 aa, Clostridium botulinum
MPVNIKNFNYNDPINNDDIIMMEPFNDPGPGTYYKAFRIIDRIWIVPERFTYGFQPDQFNASTGVFSKDVYEYYDPTYLKTDAEKDKFLKTMIKLFNRINSKPSGQRLLDMIVDAIPYLGNASTPPDKFAANVANVSINKKIIQPGAEDQIKGLMTNLIIFGPGPVLSDNFTDSMIMNGHSPISEGFGARMMIRFCPSCLNVFNNVQENKDTSIFSRRAYFADPALTLMHELIHVLHGLYGIKISNLPITPNTKEFFMQHSDPVQAEELYTFGGHDPSVISPSTDMNIYNKALQNFQDIANRLNIVSSAQGSGIDISLYKQIYKNKYDFVEDPNGKYSVDKDKFDKLYKALMFGFTETNLAGEYGIKTRYSYFSEYLPPIKTEKLLDNTIYTQNEGFNIASKNLKTEFNGQNKAVNKEAYEEISLEHLVIYRIAMCKPVMYKNTGKSEQCIIVNNEDLFFIANKDSFSKDLAKAETIAYNTQNNTIENNFSIDQLILDNDLSSGIDLPNENTEPFTNFDDIDIPVYIKQSALKKIFVDGDSLFEYLHAQTFPSNIENLQLTNSLNDALRNNNKVYTFFSTNLVEKANTVVGASLFVNWVKGVIDDFTSESTQKSTIDKVSDVSIIIPYIGPALNVGNETAKENFKNAFEIGGAAILMEFIPELIVPIVGFFTLESYVGNKGHIIMTISNALKKRDQKWTDMYGLIVSQWLSTVNTQFYTIKERMYNALNNQSQAIEKIIEDQYNRYSEEDKMNINIDFNDIDFKLNQSINLAINNIDDFINQCSISYLMNRMIPLAVKKLKDFDDNLKRDLLEYIDTNELYLLDEVNILKSKVNRHLKDSIPFDLSLYTKDTILIQVFNNYISNISSNAILSLSYRGGRLIDSSGYGATMNVGSDVIFNDIGNGQFKLNNSENSNITAHQSKFVVYDSMFDNFSINFWVRTPKYNNNDIQTYLQNEYTIISCIKNDSGWKVSIKGNRIIWTLIDVNAKSKSIFFEYSIKDNISDYINKWFSITITNDRLGNANIYINGSLKKSEKILNLDRINSSNDIDFKLINCTDTTKFVWIKDFNIFGRELNATEVSSLYWIQSSTNTLKDFWGNPLRYDTQYYLFNQGMQNIYIKYFSKASMGETAPRTNFNNAAINYQNLYLGLRFIIKKASNSRNINNDNIVREGDYIYLNIDNISDESYRVYVLVNSKEIQTQLFLAPINDDPTFYDVLQIKKYYEKTTYNCQILCEKDTKTFGLFGIGKFVKDYGYVWDTYDNYFCISQWYLRRISENINKLRLGCNWQFIPVDEGWTE

SEQ ID NO: 8 - protein, 1315 aa, Clostridium tetani
MPITINNFRYSDPVNNDTIIMMEPPYCKGLDIYYKAFKITDRIWIVPERYEFGTKPEDFNPPSSLIEGASEYYDPNYLRTDSDKDRFLQTMVKLFNRIKNNVAGEALLDKIINAIPYLGNSYSLLDKFDTNSNSVSFNLLEQDPSGATTKSAMLTNLIIFGPGPVLNKNEVRGIVLRVDNKNYFPCRDGFGSIMQMAFCPEYVPTFDNVIENITSLTIGKSKYFQDPALLLMHELIHVLHGLYGMQVSSHEIIPSKQEIYMQHTYPISAEELFTFGGQDANLISIDIKNDLYEKTLNDYKAIANKLSQVTSCNDPNIDIDSYKQIYQQKYQFDKDSNGQYIVNEDKFQILYNSIMYGFTEIELGKKFNIKTRLSYFSMNHDPVKIPNLLDDTIYNDTEGFNIESKDLKSEYKGQNMRVNTNAFRNVDGSGLVSKLIGLCKKIIPPTNIRENLYNRTASLTDLGGELCIKIKNEDLTFIAEKNSFSEEPFQDEIVSYNTKNKPLNFNYSLDKIIVDYNLQSKITLPNDRTTPVTKGIPYAPEYKSNAASTIEIHNIDDNTIYQYLYAQKSPTTLQRITMTNSVDDALINSTKIYSYFPSVISKVNQGAQGILFLQWVRDIIDDFTNESSQKTTIDKISDVSTIVPYIGPALNIVKQGYEGNFIGALETTGVVLLLEYIPEITLPVIAALSIAESSTQKEKIIKTIDNFLEKRYEKWIEVYKLVKAKWLGTVNTQFQKRSYQMYRSLEYQVDAIKKIIDYEYKIYSGPDKEQIADEINNLKNKLEEKANKAMININIFMRESSRSFLVNQMINEAKKQLLEFDTQSKNILMQYIKANSKFIGITELKKLESKINKVFSTPIPFSYSKNLDCWVDNEEDIDVILKKSTILNLDINNDIISDISGFNSSVITYPDAQLVPGINGKAIHLVNNESSEVIVHKAMDIEYNDMFNNFTVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVASSSKDVQLKNITDYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTND

SEQ ID NO: 9 - protein, 124 aa, Vibrio cholerae
MIKLKFGVFFTVLLSSAYAHGTPQNITDLCAEYHNTQIYTLNDKIFSYTESLAGKREMAIITFKNGAIFQVEVPGSQHIDSQKKAIERMKDTLRIAYLTEAKVEKLCVWNNKTPHAIAAISMAN

SEQ ID NO: 10 - protein, 258 aa, Vibrio cholerae
MVKIIFVFFIFLSSFSYANDDKLYRADSRPPDEIKQSGGLMPRGQSEYFDRGTQMNINLYDHARGTQTGFVRHDDGYVSTSISLRSAHLVGQTILSGHSTYYIYVIATAPNMFNVNDVLGAYSPHPDEQEVSALGGIPYSQIYGWYRVHFGVLDEQLHRNRGYRDRYYSNLDIAPAADGYGLAGFPPEHRAWREEPWIHHAPPGCGNAPRSSMSNTCDEKTQSLGVKFLDEYQSKVKRQIFSGYQSDIDTHNRIKDEL

SEQ ID NO: 11 - protein, 1430 aa, Artificial Sequence
  BoNT/A1(0)-CtxBCP
MGSMEFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHQLIYAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVDGIITSKTKSDDDDKTPQNITDLCAEYHNTQIHTLNDKIFSYTESLAGKREMAIITFKNGATFQVEVPGSQHIDSQKKAIERMKDTLRIAYLTEAKVEKLCVWNNKTPHAIAAISMANSGGGGSGGGGSGGGGSPRGSALNLQCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPLRKSFHHHHHH